(19) United States
(12) Patent Application Publication
(10) Pub. No.: US 2010/0256061 A1
(43) Pub. Date: Oct. 7, 2010

(54) GASTRIN COMPOUND FOR DIABETES TREATMENT

(76) Inventors: Antonio Cruz, Toronto (CA); Aleksandra Pastrak, Toronto (CA)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/529,601

(22) PCT Filed: Feb. 29, 2008
(86) PCT No.: PCT/CA08/00412

§ 371 Date: Sep. 2, 2009

Related U.S. Application Data
(60) Provisional application No. 60/904,496, filed on Mar. 2, 2007.
(60) Provisional application No. 60/937,312, filed on Jun. 27, 2007.

(51) Int. Cl. A61K 38/10; A61P 3/10

(52) U.S. Cl. 514/13

(57) ABSTRACT

The invention relates to compositions and methods for the prevention or treatment of diabetes, comprising a therapeutically effective amount of at least one gastrin compound ie gastrin G1. The gastrin compound provides beneficial effects comprising the control of haemoglobin AIc (HbA1c), fasting blood glucose, glucose levels or insulin levels. The subjects which are treated with the compositions and methods of the invention are selected based on baseline HbA1c levels or based on existing treatment with a glucose lowering agent with or without an insulin sensitivity enhancer, in particular, a metformin with or without a thiazolidinedione. The use of a gastrin compound for decreasing HbA1c levels in a diabetic subject is also proposed.

### FIELD OF THE INVENTION

The invention relates generally to compositions and methods comprising a gastrin compound, and uses thereof.

### BACKGROUND OF THE INVENTION

The insulin dependent diabetic population, including both Type 1 and Type 2 diabetics, is estimated to be approximately 4 million people in the United States and approximately 7-8 million people worldwide. The population ranges from end stage insulin dependent diabetics (transplant, severe complications) to new onset insulin diabetics. Many therapeutics and treatments have been proposed to treat and/or cure diabetes. One approach is directed at enhancing islet neogenesis. Islet neogenesis is the process by which islets are formed from precursor stem cells in the ducts of the developing fetal pancreas. Methods for treating diabetes based on islet neogenesis have been described in U.S. Pat. Nos. 5,885,956, 6,288,301 and 6,558,952.

The current invention addresses the need for additional therapies for the treatment of diabetes and related diseases, disorders, and conditions.

### SUMMARY OF THE INVENTION

Disclosed herein is the administration to a subject of at least one gastrin compound in therapeutically effective amounts to provide beneficial effects in the prevention and/or treatment of diabetes and related diseases, disorders, or conditions as well as prevention and/or treatment of complications associated with diabetes, and related diseases, disorders, or conditions.

The invention relates to compositions and methods for the prevention and/or treatment of a condition and/or disease disclosed herein comprising therapeutically effective amounts of at least one gastrin compound to provide one or more beneficial effects, in particular sustained beneficial effects.

In another aspect, the invention provides a pharmaceutical composition comprising at least one gastrin compound in therapeutically effective amounts to provide beneficial effects relative to each compound alone, preferably sustained beneficial effects, post treatment, in particular about 1 to 3, 1 to 4, 1 to 5, 1 to 6, 1 to 7, 1 to 8, 1 to 9, 1 to 10, 1 to 12, 1 to 18 or 1 to 24 months post treatment. A pharmaceutical composition may optionally comprise a pharmaceutically acceptable carrier, excipient, or vehicle.

In aspects of the invention, the compositions and methods are used to treat diabetes, in particular Type 2 diabetes, in subjects with baseline HbA1c levels greater than about 5%, 6%, 7%, 8%, 9% or 10%, in particular 5%, 6%, or 7%. In other aspects of the invention, the compositions and methods are used to treat diabetic subjects receiving one or more glucose lowering agent, an insulin sensitivity enhancer and/or insulin, in particular a metformin (e.g., metformin hydrochloride) with or without a thiazolidinedione (TZD).

In another aspect, the invention relates to a pharmaceutical composition, comprising at least one gastrin compound in therapeutically effective amounts to provide beneficial effects and at least one additional pharmaceutically acceptable carrier, excipient, or vehicle.

The invention also contemplates a pharmaceutical composition in containers and intended for administration to a subject to provide one or more beneficial effects, preferably sustained beneficial effects, comprising at least one gastrin compound in therapeutically effective amounts, together with pharmaceutically acceptable carriers, excipients, or vehicles.

In aspects, a pharmaceutical composition of the invention comprises at least one gastrin compound in therapeutically effective amounts to control haemoglobin A1c (HbA1c), fasting blood glucose, glucose levels and/or insulin levels. In particular aspects of the invention, a pharmaceutical composition of the invention comprises at least one gastrin compound in therapeutically effective amounts to decrease HbA1c levels by at least about 0.1%, 0.25%, 0.4%, 0.43%, 0.45%, 0.5%, 0.6%, 0.7%, 0.75%, 0.8%, 0.85%, 0.9%, 0.93%, 0.94%, 0.97%, 0.98%, 1%, 1.05%, 1.09%, 1.1%, 1.12%, 1.14%, 1.15%, 1.18%, 1.21%, 1.2%, 1.5%, 2%, 5%, 10%, 20%, 30%, 33%, 35%, 40%, 45%, or 50%, in particular to decrease in HbA1c levels by at least about 0.4%, 0.5%, 0.75%, 0.8%, 0.9%, 0.95%, 1%, 1.5% or 2%, more particularly between about 0.5% to 0.8%, 0.75% to 2%, 0.8% to 2%, 0.9% to 1.5%, 0.95% to 1.5%, 1% to 1.5% or 0.97% to 1.21% versus baseline; increase insulin levels by at least about 0.05%, 5%, 10%, 25%, 50%, 70%, 75%, 80%, 90%, or 95%; and/or increase C-peptide levels by at least about 0.05%, 1%, 5%, 15%, 20%, 25%, 30% or 35%. In an embodiment, the therapeutically effective amounts are amounts effective to decrease HbA1c levels by at least about 0.4%, 0.5%, 0.75%, 0.8%, 0.85%, 0.9%, 0.95%, 0.97%, 1%, 1.2%, 1.4%, 1.5% or 2% post-treatment in Type 2 diabetes patients with baseline HbA1c levels greater than about 5%, 6%, 7%, 8%, 9% or 10%, in particular 5%, 6%, or 7%.

In an aspect the invention relates to a medicinal composition of active principles (i.e. one or more gastrin compound), having a therapeutic action for the treatment of Type 1 diabetes.

In another aspect the invention relates to a medicinal composition of active principles (i.e. one or more gastrin compound), having a therapeutic action for the treatment of Type 2 diabetes.

The invention still further contemplates methods for preparing compositions of the invention that result in compositions with one or more beneficial effects, preferably sustained beneficial effects. In an aspect of the invention, a method is provided for preparing a pharmaceutical composition of at least one gastrin compound in therapeutically effective amounts adapted to provide one or more beneficial effects post-treatment, in particular 1 to 3, 1 to 4, 1 to 5, 1 to 6, 1 to 7, 1 to 8, 1 to 9, 1 to 10, 1 to 11, or 1 to 12 months post-treatment, comprising preparing a composition comprising at least one gastrin compound, and a pharmaceutically acceptable carrier, excipient, or vehicle. The beneficial effects can comprise therapeutically effective amounts of at least one gastrin compound to control haemoglobin A1c (HbA1c), fasting blood glucose, glucose levels and/or insulin levels, in particular for 1 to 3, 1 to 4, 1 to 5, 1 to 6, 1 to 7, 1 to 8, or 1 to 9 months post-treatment.

The invention relates to a treatment for preventing and/or treating a condition and/or disease disclosed herein in a subject comprising administering to the subject therapeutically effective amounts of at least one gastrin compound, in particular to provide one or more beneficial effects following treatment, in particular 1 to 3 months, 1 to 4 months, 1 to 5 months, 1 to 6 months, 1 to 9 months, 1 to 12 months, 1 to 18 months, or 1 to 24 months post treatment. In an aspect of the invention, the subject is administered at least one gastrin compound for about 1 to 4 weeks, 1 to 5 weeks, 2 to 4 weeks, 2 to 6 weeks, 2 to 8 weeks, or 2 to 12 weeks. In aspects of the invention, the treatment is stopped for about 1 to 12 months, 1 to 9 months, 1 to 8 months, 1 to 7 months, 1 to 6 month, 1 to 5 months, 1 to 4 months, 3 to 12 months, 3 to 9 months, 3 to 6 months, 6 to 9 months, 6 to 12 months, 6 to 18 months, 6 to 24 months, 9 to 12 months or 12 to 18 months, following administration, in particular 3 to 6 months or 3 to 9 months, more particularly 6 months, following administration.

In an aspect of the invention a treatment is provided for preventing and/or treating diabetes in a subject comprising administering to the subject therapeutically effective amounts of at least one gastrin compound to control haemoglobin A1c (HbA1c), fasting blood glucose, glucose levels and/or insulin levels. In a particular aspect of the invention a treatment is provided for preventing and/or treating Type 2 diabetes in a subject comprising administering to the subject therapeutically effective amounts of at least one gastrin compound to decrease HbA1c levels by at least about 0.1%, 0.25%, 0.4%, 0.43%, 0.45%, 0.5%, 0.6%, 0.7%, 0.75%, 0.8%, 0.85%, 0.9%, 0.93%, 0.94%, 0.97%, 0.98%, 1%, 1.05%, 1.09%, 1.1%, 1.12%, 1.14%, 1.15%, 1.18%, 1.2%, 1.21%, 1.5%, or 2%, in particular at least about 0.5%, 0.75%, 0.8%, 0.85%, 0.9%, 0.95%, 0.97%, 1%, 1.5% or 2%; increase insulin levels by at least about 50%, 70%, 75%, 80%, 90%, or 95%; and/or increase C-peptide levels by at least about 15%, 20%, 25%, 26%, 30%, 34%, 35%, 40%, 50%, 60%, 70%, 80%, or 90%.

In an embodiment a treatment is provided for preventing and/or treating Type 2 diabetes in a subject with baseline HbA1c levels greater than about 5%, 6%, 7%, 8%, 9% or 10%, in particular 5%, 6%, or 7%, comprising administering to the subject therapeutically effective amounts of at least one gastrin compound to decrease HbA1c levels by at least about 0.1%, 0.25%, 0.4%, 0.43%, 0.45%, 0.5%, 0.6%, 0.7%, 0.75%, 0.8%, 0.85%, 0.9%, 0.93%, 0.94%, 0.97%, 0.98%, 1%, 1.05%, 1.09%, 1.1%, 1.12%, 1.14%, 1.15%, 1.18%, 1.2%, 1.21%, 1.5%, or 2% post-treatment. In other embodiments the subject is a Type 2 diabetes patient using one or more glucose lowering agent, an insulin and/or an insulin sensitivity enhancer, in particular a metformin with or without thiazolidinediones (TZDs).

The invention further relates to the use of at least one gastrin compound or a composition of the invention for preventing, delaying progression of, and/or ameliorating disease severity, disease symptoms, and/or periodicity of recurrence of a condition and/or disease disclosed herein. In particular, the invention relates to the prevention, delay of progression, and/or treatment, in a subject suffering from diabetes, more particularly Type 2 diabetes, using at least one gastrin compound or a composition of the invention. In embodiments, the condition/and or disease is Type 2 diabetes and the subject has baseline HbA1c levels greater than about 5%, 6%, 7%, 8%, 9% or 10%, in particular 5%, 6%, or 7%. In an embodiment the subject uses one or more glucose lowering agent and an insulin sensitivity enhancer, in particular a metformin with or without thiazolidinediones (TZDs).

In an aspect, the invention provides a method for the prevention and/or intervention of diabetes in a subject, in particular a subject with baseline HbA1c levels greater than about 5%, 6%, 7%, 8%, 9% or 10%, in particular 5%, 6%, or 7%, comprising administration of therapeutically effective amounts of at least one gastrin compound or a composition of the invention. A gastrin compound or a composition may be directly administered to a subject or contacted with cells (e.g. stem cells or progenitor cells) and administered to a subject. In other aspects, the invention provides a method for the prevention and/or intervention of a condition and/or disease disclosed herein in a subject comprising administration of at least one gastrin compound to a subject in need thereof to provide beneficial effects including controlling haemoglobin A1c (HbA1c), fasting blood glucose, glucose levels and/or insulin levels. In a particular aspect of the invention the beneficial effects include decreasing HbA1c levels by at least about 0.1%, 0.25%, 0.4%, 0.43%, 0.45%, 0.5%, 0.6%, 0.7%, 0.75%, 0.8%, 0.85%, 0.9%, 0.93%, 0.94%, 0.97%, 0.98%, 1%, 1.05%, 1.09%, 1.1%, 1.12%, 1.14%, 1.15%, 1.18%, 1.21%, 1.2%, 1.5%, or 2%, in particular at least about 0.5%, 0.75%, 0.8%, 0.85%, 0.9%, 0.97%, 1%, 1.5%, or 2%; increasing insulin levels by at least about 50%, 70%, 75%, 80%, 90%, or 95%; and/or increasing C-peptide levels by at least about 15%, 20%, 25%, 26%, 30%, 34%, 35%, 40%, 50%, 60%, 70%, 80%, or 90%. In a particular aspect, the diabetic subject has baseline HbA1c levels greater than about 5%, 6%, or 7%.

In aspects of the invention, a therapeutically effective amount of a glucose lowering agent and/or an insulin sensitivity enhancer is also administered, separately or together with a gastrin compound(s) to the subject. Thus, in methods and treatments of the invention the subject can be a Type 2 diabetes patient using one or more glucose lowering agent and an insulin sensitivity enhancer, in particular a metformin with or without thiazolidinediones (TZDs).

The invention provides in some aspects methods for the potentiation of a glucose lowering agent and/or an insulin sensitivity enhancer in the treatment of Type 2 diabetes in a subject comprising co-administering therapeutically effective amounts of at least one gastrin compound and at least one glucose lowering agent and/or insulin sensitivity enhancer to the subject.

In another aspect, the invention relates to a method for treating diabetes in a patient in need thereof by administering at least one gastrin compound or a composition comprising at least one gastrin compound, in amount(s) sufficient to stimulate insulin production in existing islet cells.

In aspects of the invention, a therapeutically effective amount of an insulin or insulin analog is also administered, separately or together with a gastrin compound(s) to the subject.

In aspects of the invention, a therapeutically effective amount of an immunosuppressive agent is also administered, separately or together with a gastrin compound(s) to the subject.

In aspects of the invention, a therapeutically effective amount of an insulin secretagogue is also administered, separately or together with a gastrin compound(s) to the subject.

In aspects of the invention, a therapeutically effective amount of an antiobesity or appetite regulating agent is also administered, separately or together with a gastrin compound(s, to the subject.

The invention further contemplates use of at least one gastrin compound for the prevention and/or treatment of a condition and/or disease, or for the manufacture of a medicament for the prevention and/or treatment of a condition and/or disease. In particular, the invention contemplates the use of therapeutically effective amounts of at least one gastrin compound for decreasing HbA1c levels by at least about 0.1%, 0.25%, 0.4%, 0.43%, 0.45%, 0.5%, 0.6%, 0.7%, 0.75%, 0.8%, 0.85%, 0.9%, 0.93%, 0.94%, 0.97%, 0.98%, 1%, 1.05%, 1.09%, 1.1%, 1.12%, 1.14%, 1.15%, 1.18%, 1.21%, 1.2%, 1.5%, 2%, 5%, 10%, 20%, 30%, 33%, 35%, 40%, 45%, or 50%; increasing insulin levels by at least about 50%, 70%, 75%, 80%, 90%, or 95%; and/or increasing C-peptide levels by at least about 15%, 20%, 25%, 26%, 30%, 34%, 35%, 40%, 50%, 60%, 70%, 80%, or 90%, in a subject, in particular a subject with Type 2 diabetes and baseline HbA1c levels greater than about 5%, 6%, 7%, 8%, 9% or 10%, in particular 5%, 6%, or 7%. In particular, the invention contemplates the use of therapeutically effective amounts of at least one gastrin compound for the preparation of one or more medicament for decreasing HbA1c levels by at least about 0.1%, 0.25%, 0.4%, 0.43%, 0.45%, 0.5%, 0.6%, 0.7%, 0.75%, 0.8%, 0.85%, 0.9%, 0.93%, 0.94%, 0.97%, 0.98%, 1%, 1.05%, 1.09%, 1.1%, 1.12%, 1.14%, 1.15%, 1.18%, 1.21%, 1.2%, 1.5%, 2%, 5%, 10%, 20%, 30%, 33%, 35%, 40%, 45%, or 50%; increasing insulin levels by at least about 50%, 70%, 75%, 80%, 90%, or 95%; and/or increasing C-peptide levels by at least about 15%, 20%, 25%, 26%, 30%, 34%, 35%, 40%, 50%, 60%, 70%, 80%, or 90%, in a subject, in particular a subject with Type 2 diabetes and baseline HbA1c levels greater than about 5%, 6%, 7%, 8%, 9% or 10%, in particular 5%, 6%, or 7%. In particular, the invention contemplates the use of therapeutically effective amounts of at least one gastrin compound for decreasing HbA1c levels by at least about 0.1%, 0.25%, 0.4%, 0.43%, 0.45%, 0.5%, 0.6%, 0.7%, 0.75%, 0.8%, 0.85%, 0.9%, 0.93%, 0.94%, 0.97%, 0.98%, 1%, 1.05%, 1.09%, 1.1%, 1.12%, 1.14%, 1.15%, 1.18%, 1.21%, 1.2%, 1.5%, 2%, 5%, 10%, 20%, 30%, 33%, 35%, 40%, 45%, or 50%; in particular about 0.4%, 0.5%, 0.75%, 0.8%, 0.9%, 0.95%, 0.97%, 1%, 1.5% or 2%, post-treatment in a subject with Type 2 diabetes. In particular, the invention contemplates the use of therapeutically effective amounts of at least one gastrin compound for decreasing HbA1c levels by at least about 0.4%, 0.5%, 0.75%, 0.8%, 0.9%, 0.95%, 0.97%, 1%, 1.5% or 2% post-treatment in a subject with Type 2 diabetes, or for the preparation of one or more medicament for decreasing HbA1c levels by at least about 0.4%, 0.5%, 0.75%, 0.8%, 0.9%, 0.95%, 0.97%, 1%, 1.5% or 2% post-treatment in a subject with Type 2 diabetes.

The invention additionally provides uses of a pharmaceutical composition of the invention for the prevention and/or treatment of conditions and/or diseases disclosed herein, or in the preparation of medicaments for the prevention and/or treatment of conditions and/or diseases disclosed herein. The medicaments may provide beneficial effects, preferably sustained beneficial effects following treatment.

The invention also provides a kit comprising at least one gastrin compound, and a pharmaceutical composition of the invention in kit form.

In selected compositions, methods, treatments, uses and kits of the invention that provide beneficial effects, including controlling haemoglobin A1c (HbA1c), fasting blood glucose, glucose levels and/or insulin levels, more particularly decreased HbA1c levels by at least about 0.1%, 0.25%, 0.4%, 0.43%, 0.45%, 0.5%, 0.6%, 0.7%, 0.75%, 0.8%, 0.85%, 0.9%, 0.93%, 0.94%, 0.97%, 0.98%, 1%, 1.05%, 1.09%, 1.1%, 1.12%, 1.14%, 1.15%, 1.18%, 1.21%, 1.2%, 1.5%, or 2%; increased insulin levels by at least about 50%, 70%, 75%, 80%, 90%, or 95%; and/or increased C-peptide levels by at least about 15%, 20%, 25%, 26%, 30%, 34%, 35%, 40%, 50%, 60%, 70%, 80%, or 90%, the gastrin compound is a gastrin analogue, in particular gastrin-17(leu) of SEQ ID NO. 4.

These and other aspects, features, and advantages of the present invention should be apparent to those skilled in the art from the following detailed description and accompanying drawings.

### DESCRIPTION OF THE DRAWINGS

The invention will be better understood with reference to the drawings in which:

FIG. 1 are graphs showing HbA1c (%) and changes in HbA1c (%) from baseline to post-treatment in all subjects in a Type 2 diabetes study.

FIG. 2 are graphs showing HbA1c (%) levels and changes in HbA1c (%) from baseline to post-treatment in all subjects with baseline HbA1c≧7% in a Type 2 diabetes study.

FIG. 3 are graphs showing fasting glucose (mg/dL) from baseline to post-treatment in all subjects or subjects with baseline HbA1c≧7% in a Type 2 diabetes study.

FIG. 4 are graphs showing Oral Glucose Tolerance Test (OGTT) glucose AUC (g·min/dL) from baseline to post-treatment in all subjects or subjects with baseline HbA1c≧7% in a Type 2 diabetes study.

FIG. 5 are graphs showing OGTT insulin AUC (U·min/L) from baseline to post-treatment in all subjects or subjects with baseline HbA1c≧7% in a Type 2 diabetes study.

FIG. 6 are graphs showing OGTT insulin:glucose ratio (AUCs) (×10⁻⁹) from baseline to post-treatment in all subjects or subjects with baseline HbA1c≧7% in a Type 2 diabetes study.

FIG. 7 are graphs showing fasting proinsulin:insulin ratio from baseline to post-treatment in all subjects or subjects with baseline HbA1c≧7% in a Type 2 diabetes study.

FIG. 8 are graphs showing arginine-stimulated C-peptide (ng·min/ml) from baseline to post-treatment in all subjects or subjects with baseline HbA1c≧7% in a Type 2 diabetes study.

FIG. 9 are graphs showing OGTT C-peptide (ng·min/ml) from baseline to post-treatment in all subjects or subjects with baseline HbA1c≧7% in a Type 2 diabetes study.

FIG. 10 are graphs showing the change in OGTT insulin AUC from baseline and change in OGTT C-peptide AUC from baseline in E1+G1 patients with an HbA1c drop≧1.0% from baseline in a Type 2 diabetes study.

FIG. 11 is a graph showing changes in insulin usage (%) post-treatment in patients receiving placebo, E1+G1 non-responders and E1+G1 responders in a Type I diabetes study.

FIG. 12 is a graph showing changes in HbA_{1c} (%) in post-treatment Month 2 in patients receiving placebo, E1+G1 non-responders and E1+G1 responders in a Type 1 diabetes study.

FIG. 13 are graphs showing HbA_{1C} (%) and changes in HbA_{1C} (%) from baseline to post-treatment in all subjects in a study with Type 2 diabetes patients.

FIG. 14 are graphs showing HbA_{1C} (%) levels and changes in HbA_{1C} (%) from baseline to post-treatment in subjects with baseline HbA_{1C}≧7% in a study with Type 2 diabetes patients.

FIG. 15 are graphs showing average fasting glucose (mg/dL) levels in post-treatment months and changes from baseline to post-treatment in all subjects in a study with Type 2 diabetes patients.

FIG. 16 are graphs showing average fasting glucose (mg/dL) levels in post-treatment months and changes from baseline to post-treatment in subjects with baseline HbA_{1C}≧7% in a study with Type 2 diabetes patients.

FIG. 17 are graphs showing average oral glucose tolerance test (OGTT) glucose AUCs (g·min/dL) in post-treatment months in all subjects or subjects with baseline HbA1c≧7% in a study with Type 2 diabetes patients.

FIG. 18 are graphs showing average OGTT insulin AUCs (U·min/L) in post-treatment months in all subjects or subjects with baseline HbA1c≧7% in a study with Type 2 diabetes patients.

FIG. 19 are graphs showing average OGTT insulin AUC:glucose AUC ratios in post-treatment months in all subjects or subjects with baseline HbA1c≧7% in a study with Type 2 diabetes patients.

FIG. 20 are graphs showing average arginine-stimulated C-peptide AUC (ng·min/ml) levels in post-treatment months in all subjects or subjects with baseline HbA1c≧7% in a study with Type 2 diabetes patients.

FIG. 21 are graphs showing average OGTT C-peptide AUC (ng·min/ml) levels in post-treatment months in all subjects or subjects with baseline HbA1c≧7% in a study with Type 2 diabetes patients.

FIG. 22 are graphs showing average change (%) in OGTT insulin AUCs from baseline and average changes (%) in OGTT C-peptide AUCs from baseline in E1+G1 patients with an HbA1c drop≧1.0% from baseline vs. all placebo subjects in a study with Type 2 diabetes patients.

FIG. 23 are graphs showing average changes (%) in insulin usage from baseline in all subjects or subjects with greater than 20% decrease in daytime insulin usage during post-treatment months in a study with Type 1 diabetes patients.

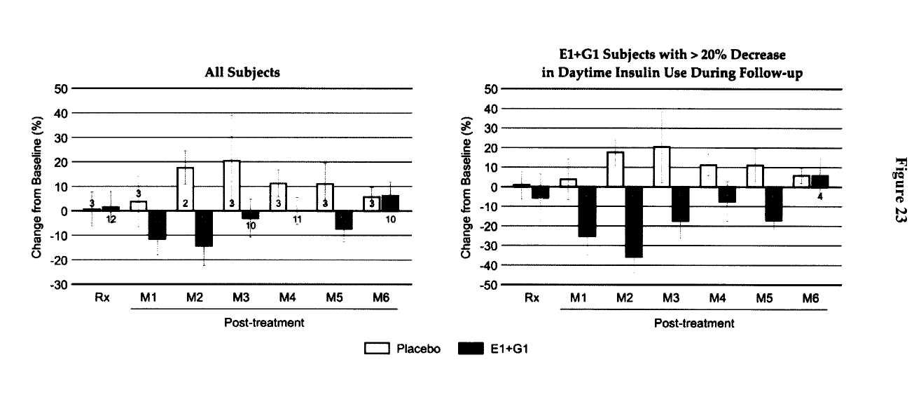

FIG. 24 are graphs showing average changes in HbA_{1C} (%) from baseline in all subjects or subjects with greater than 20% decrease in daytime insulin usage during post-treatment months in a study with Type 1 diabetes patients.

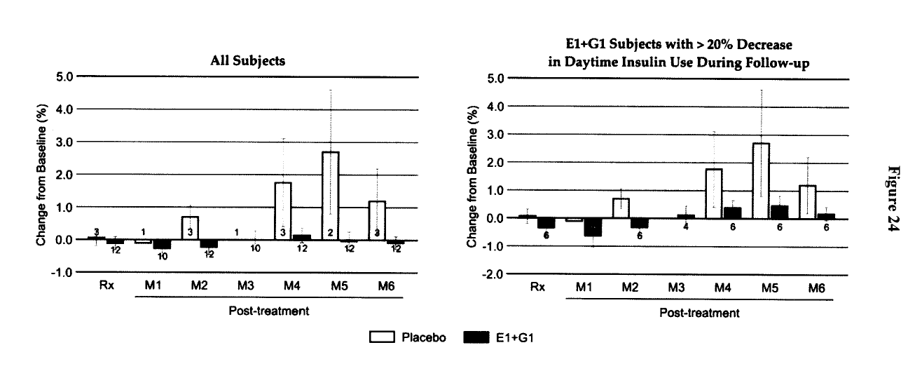

### GLOSSARY

Unless defined otherwise, all technical and scientific terms used herein have the same meaning as commonly understood by one of ordinary skill in the art to which this invention belongs.

As used herein, the terms “comprising,” “including,” and “such as” are used in their open and non-limiting sense.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made.

Further, it is to be understood that “a,” “an,” and “the” include plural referents unless the content clearly dictates otherwise. For example, reference to “a compound” includes a mixture of two or more compounds. Thus, the phrase “a gastrin compound”, as used herein can also mean “one or more gastrin compound” or “at least one gastrin compound”.

Selected compounds described herein contain one or more asymmetric centers and may give rise to enantiomers, diasteriomers, and other stereoisomeric forms which may be defined in terms of absolute stereochemistry as (R)— or (S)—. Therefore, the invention includes all such possible diasteriomers and enantiomers as well as their racemic and optically pure forms. Optically active (R)- and (S)-isomers may be prepared using chiral synthons or chiral reagents, or resolved using conventional techniques. When the compounds described herein contain centers of geometric asymmetry, and unless specified otherwise, it is intended that the compounds include both E and A geometric isomers. All tautomeric forms are intended to be included within the scope of the invention.

The terms “administering” and “administration” refer to the process by which a therapeutically effective amount of a compound or a composition contemplated herein is delivered to a subject for prevention and/or treatment purposes. Compositions are administered in accordance with good medical practices taking into account the subject's clinical condition, the site and method of administration, dosage, patient age, sex, body weight, and other factors known to physicians.

“Administering in combination” or “in combination” means that the active ingredients are administered concurrently to a patient being treated. When administered in combination each component may be administered at the same time, or sequentially in any order at different points in time. Therefore, each component may be administered separately, but sufficiently close in time to provide the desired effect, in particular a beneficial, complementary, additive, or synergistic effect. The first compound may be administered in a regimen which additionally comprises treatment with an additional compound. In certain embodiments, the term refers to administration of one or more gastrin compound and an additional therapeutic agent to a patient within 1 day, 2 days, 3 days, four days, five days, six days, 1 week, 2 weeks, 3 weeks, 4 weeks, 8 weeks, 12, weeks, 18 weeks, or one year, preferably within 1 to 5 days, 1 to 4 days, 1 to 3 days, or 1 to 2 days, including separate administration of the medicaments each containing one of the compounds as well as simultaneous administration whether or not the compounds are combined in one formulation or whether they are two separate formulations.

The terms “subject”, “individual” or “patient” refer to an animal including a warm-blooded animal such as a mammal, which is afflicted with or suspected of having or being pre-disposed to a condition and/or disease as disclosed herein. Preferably, the terms refer to a human. The terms also include domestic animals bred for food, sport, or as pets, including horses, cows, sheep, poultry, fish, pigs, cats, dogs, and zoo animals. The methods herein for use on subjects/individuals/patients contemplate prophylactic as well as curative use. Typical subjects for treatment include persons susceptible to, suffering from or that have suffered a condition and/or disease disclosed herein. In aspects of the invention, a subject has Type 1 diabetes. In aspects of the invention, a subject has Type 2 diabetes. In particular aspects of the invention, a subject has baseline HbA1c levels greater than about 5%, 6%, 7%, 8%, 9% or 10%, in particular 5%, 6%, or 7%. In aspects of the invention, the subject is receiving one or more glucose lowering agent, an insulin sensitivity enhancer and/or an insulin. In particular aspects of the invention, the subject has Type 2 diabetes and is receiving a glucose lowering agent and/or an insulin sensitivity enhancer, in particular a metformin with or without thiazolidinediones (TZDs).

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. The use of such media and agents for an active substance is well known in the art.

“Pharmaceutically acceptable salt(s),” means a salt that is pharmaceutically acceptable and has the desired pharmacological properties. By pharmaceutically acceptable salts is meant those salts which are suitable for use in contact with the tissues of a subject or patient without undue toxicity, irritation, allergic response and the like, and are commensurate with a reasonable benefit/risk ratio. Pharmaceutically acceptable salts are described for example, in S. M. Berge, et al., J. Pharmaceutical Sciences, 1977, 66:1. Suitable salts include salts that may be formed where acidic protons in the compounds are capable of reacting with inorganic or organic bases. Suitable inorganic salts include those formed with alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N-methylglucamine, and the like. Suitable salts also include acid addition salts formed with inorganic acids (e.g. hydrochloride and hydrobromic acids) and organic acids (e.g. acetic acid, citric acid, maleic acid, and the alkane- and arene-sulfonic acids such as methanesulfonic acid and benezenesulfonic acid). When there are two acidic groups present, a pharmaceutically acceptable salt may be a mono-acid-mono-salt or a di-salt; and similarly where there are more than two acidic groups present, some or all of such groups can be salified.

The terms “preventing and/or treating”, “prevention and/or treatment”, or “prevention and/or intervention” refer to the administration to a subject of biologically active agents either before or after onset of a condition and/or disease. A treatment may be either performed in an acute or chronic way. In particular, prevention includes the management and care of a subject at risk of developing a condition and/or disease disclosed herein prior to the clinical onset of the condition and/or disease. Treatment or intervention refers to the management and care of a subject at diagnosis or later. An objective of prevention, treatment, or intervention is to combat the condition and/or disease and includes administration of the active compounds to prevent or delay the onset of the symptoms or complications, or alleviating the symptoms or complications, or eliminating or partially eliminating the condition and/or disease.

A “beneficial effect” refers to favourable pharmacological and/or therapeutic effects, and/or improved pharmacokinetic properties and biological activity of at least one gastrin compound, or composition thereof. A beneficial effect or sustained beneficial effect may manifest as one or more of increased C-peptide levels, increased insulin levels, decreased HBA1c levels, about normal or reduced blood glucose levels, decreased insulin dependence or delivery, and reduction in insulin use in a subject. In aspects of the invention, beneficial effects include but are not limited to the following: reduced or absent islet inflammation, decreased disease progression, decreased or alleviated disease symptoms, increased survival, or elimination or partial elimination of a condition and/or disease.

In particular aspects, the beneficial effect is a “sustained beneficial effect” where the beneficial effect is sustained for a prolonged period of time after termination of treatment. In an embodiment, one or more of the aforementioned effects are sustained for a prolonged period of time after termination of treatment. A beneficial effect may be sustained for at least about 2, 4, 6, 7, 8, 9, 10, 11, or 12 weeks, 2 to 4 weeks, 2 to 6 weeks, 2 to 8 weeks, 2 to 12 weeks, 2 to 24 weeks, 2 weeks to 12 months, 2 weeks to 18 months, 1 to 2 months, 1 to 3 months, 1 to 4 months, 1 to 5 months, 1 to 6 months, 1 to 7 months, 1 to 8 months, 1 to 9 months, 1 to 12 months, 1 to 18 months, or 1 to 24 months following treatment. The period of time a beneficial effect is sustained may correlate with the duration and timing of the treatment. A subject may be treated continuously for about 1 to 4 weeks, 1 to 5 weeks, 2 to 4 weeks, 2 to 6 weeks, 2 to 8 weeks, 2 to 12 weeks, 2 to 16 weeks, 1 to 2 months, 1 to 3 months, 1 to 4 months, 1 to 5 months, 1 to 6 months, 2 weeks to 6 months, 2 weeks to 12 months, or periodically. In an aspect, a subject is treated daily for at least 2, 3, 4, 5 or 6 weeks, more particularly 4 weeks.

The beneficial effect may be a statistically significant effect in terms of statistical analysis of an effect of the compounds versus the effects of each of the compounds or two compounds alone. “Statistically significant” or “significantly different” effects or levels with the compounds compared with each compound alone or without the compounds may represent levels that are higher or lower than a standard. In embodiments of the invention, the difference may be 0.5, 1, 1.5, 2, 3, 4, 5, 6, 7, 8, 9, 10, 15, 20, 30, 40 or 50 times higher or lower compared with the effect obtained with each compound alone or in the absence of the compounds.

A “medicament” refers to a pharmaceutical composition suitable for administration of pharmaceutically active compound(s) (e.g. one or more gastrin compound) to a patient.

“Therapeutically effective amount” relates to the amount or dose of active compounds (e.g. gastrin compound) or compositions of the invention that will lead to one or more beneficial effects, preferably one or more sustained beneficial effects. A “therapeutically effective amount” can provide a dosage which is sufficient in order for prevention and/or treatment of a condition and/or disease in a subject to be effective compared with no treatment.

“Suboptimal dose” or suboptimal dosage” refers to a dose or dosage of one or more active compound which is less than the optimal dose or dosage for that compound when used in monotherapy.

The term “potentiation” refers to an increase of a corresponding pharmacological activity or therapeutic effect. Potentiation of one component of a combination by co-administration of the other component means that an effect is being achieved that is greater than that achieved with one component alone.

The term “substantial similarity” or “substantial sequence similarity,” when referring to a polypeptide indicates that, when optimally aligned with another polypeptide there is a percent sequence identity in at least about 50%, more preferably 60% of the amino acid residues, usually at least about 70%, more usually at least about 80%, preferably at least about 90%, and more preferably at least about 95-98% of the amino acid residues.

“Percent sequence identity” or “sequence identity” refers to the percentage of amino acid residues in a candidate sequence that are identical with the amino acid residues in a polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various conventional ways, for instance, using publicly available computer software including the GCG program package (Devereux J. et al., Nucleic Acids Research 12(1): 387, 1984); BLASTP, BLASTN, and FASTA, Gap or Bestfit (Wisconsin Package Version 10.0, Genetics Computer Group (C4CG), Madison, Wis.; Pearson, Methods Enzymol. 183: 63-98, 1990; Pearson, Methods Mol. Bio. 276: 71-84, 1998). The BLAST programs are publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

An “analog” refers to a polypeptide wherein one or more amino acid residues of a parent or wild-type polypeptide have been substituted by another amino acid residue, one or more amino acid residues of a parent or wild-type polypeptide have been inverted, one or more amino acid residues of the parent or wild-type polypeptide have been deleted, and/or one or more amino acid residues have been added to the parent or wild-type polypeptide. Such an addition, substitution, deletion, and/or inversion may be at either of the N-terminal or C-terminal end or within the parent or wild-type polypeptide, or a combination thereof. Typically “an analog” is a peptide wherein 6 or less amino acids have been substituted and/or added and/or deleted from the parent or wild-type peptide, more preferably a peptide wherein 3 or less amino acids have been substituted and/or added and/or deleted from the parent or wild-type polypeptide, and most preferably, a peptide wherein one amino acid has been substituted and/or added and/or deleted from the parent or wild-type polypeptide.

Mutations may be introduced into a polypeptide by standard methods, such as site-directed mutagenesis and PCR-mediated mutagenesis. Conservative substitutions can be made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed.

A “derivative” refers to a polypeptide in which one or more of the amino acid residues of a parent polypeptide have been chemically modified. Derivatives may be obtained by chemically modifying one or more amino acid residues of the parent polypeptide or analog thereof, for instance by alkylation, acylation, glycosylation, pegylation, ester formation, deamidation, amide formation, or by introducing a lipophilic functionality. In aspects of the invention, “a derivative” designates a peptide or analogue thereof which is chemically modified by introducing an ester, alkyl or lipophilic functionality on one or more amino acid residues of the peptide or analogue thereof.

A “chimeric polypeptide” comprises all or part (preferably biologically active) of a selected polypeptide operably linked to a heterologous polypeptide (i.e., a polypeptide other than the selected polypeptide). Within the fusion polypeptide, the term “operably linked” is intended to indicate that a selected polypeptide and the heterologous polypeptide are fused in-frame to each other. The heterologous polypeptide can be fused to the N-terminus or C-terminus of a selected polypeptide. Chimeric and fusion proteins can be produced by standard recombinant DNA techniques.

A “gastrin/CCK receptor” refers to a member of the G-protein-coupled receptor family that displays a characteristic binding affinity for a cholecystokinin (CCK) including without limitation CCK-8, desulfated CCK-8, CCK-33, CCK-4, or gastrins including without limitation desulfated or sulfated gastrin-17, or pentagastrin, or other CCK or gastrin analogues or family members. Examples of gastrin/CCK receptor proteins are gastrin/CCK_{A} receptors and gastrin/CCK_{B} receptors, in particular a gastrin/CCK_{B} receptor.

A “gastrin compound” refers to any compound, including peptides and non-peptide compounds, which fully or partially associate with and/or activate a gastrin/CCK receptor and/or increase gastrin secretion. In aspects of the invention, a gastrin compound is selected that has a suitable IC₅₀, for example an IC₅₀ of about ˜0.7 nM, at a gastrin/CCK receptor, in particular a gastrin/CCK_{B} receptor, as measured by methods known in the art (see Singh et al (1995) supra, and Kopin et al (1995) supra describing in vitro cell growth assays and receptor binding assays). A gastrin compound may also be selected based on other criteria such as activity, half-life etc.

The term “gastrin compound” encompasses compounds that provide at least one beneficial effect. In aspects of the invention a gastrin compound is selected such that when it is administered to a diabetic subject haemoglobin A1c (HbA1c), fasting blood glucose, glucose levels and/or insulin levels are controlled, neogenesis of insulin-producing pancreatic islet cells is induced, levels of C-peptide are increased, levels of insulin are increased, HBA1c levels are decreased, and/or blood glucose levels are reduced or at or about normal. In particular aspects, a gastrin compound is selected such that when it is administered to a diabetic subject (e.g. a Type 2 diabetic subject), there is a decrease in HbA1c levels by at least about 0.1%, 0.25%, 0.4%, 0.43%, 0.45%, 0.5%, 0.6%, 0.7%, 0.75%, 0.8%, 0.85%, 0.9%, 0.93%, 0.94%, 0.97%, 0.98%, 1%, 1.05%, 1.09%, 1.1%, 1.12%, 1.14%, 1.15%, 1.18%, 1.21%, 1.2%, 1.5%, 2%, 5%, 10%, 20%, 30%, 33%, 35%, 40%, 45%, or 50%; an increase in insulin levels by at least about 0.5%, 5%, 10%, 25%, 50%, 70%, 75%, 80%, 90%, or 95%, and/or an increase in C-peptide levels by at least about 0.05%, 5%, 15%, 20%, 25%, 26%, 30%, 34%, 35%, 40%, 50%, 60%, 70%, 80%, or 90%. In other aspects of the invention a gastrin compound is selected such that in a diabetic subject there is a decrease in insulin usage. In more particular aspects, a gastrin compound is selected such that when it is administered to a Type 2 diabetic subject, preferably a subject having baseline HbA1c levels greater than about 5%, 6%, 7%, 8%, 9% or 10%, in particular 5%, 6%, or 7%, and/or a subject receiving a glucose lowering agent and/or an insulin sensitivity enhancer, in particular a metformin with or without TZDs, there is a decrease in HbA1c levels by at least about 0.1%, 0.25%, 0.4%, 0.43%, 0.45%, 0.5%, 0.6%, 0.7%, 0.75%, 0.8%, 0.85%, 0.9%, 0.93%, 0.94%, 0.97%, 0.98%, 1%, 1.05%, 1.09%, 1.1%, 1.12%, 1.14%, 1.15%, 1.18%, 1.21%, 1.2%, 1.5%, or 2%; an increase in insulin levels by at least about 70%, 75%, 80%, 90%, or 95%, and/or an increase in C-peptide levels by at least about 15%, 20%, 25%, 30% or 35%. In aspects of the invention the term includes any gastrin compound that demonstrates additive, synergistic or complementary activity with one or more glucose lowering agent and/or insulin sensitivity enhancer, in particular a metformin with or without TZDs.

The term “gastrin compound” includes analogs, derivatives, fragments and modifications of a wild-type gastrin and chimeric polypeptides comprising gastrin. In aspects of the invention a gastrin compound includes a polypeptide that shares substantial sequence similarity with a mammalian gastrin and possesses some or all of the biological activity of a mammalian gastrin. In certain aspects, a gastrin compound may be an active analog, fragment or other modification which, for example, share amino acid sequence similarity with an endogenous mammalian gastrin, for example, share 60%, 70%, 80%, 90%, 95%, 98%, or 99% identity. The term also includes a free base, free acid, salt or pharmaceutically acceptable salt, hydrate, ester, amide, enantiomer, isomer, tautomer, polymorph, metabolite or prodrug of a gastrin compound.

A “gastrin compound” includes, without limitation, the various forms of gastrin, such as gastrin 71, gastrin 52, gastrin 34 (big gastrin), gastrin 17 (little gastrin), gastrin 14, and gastrin 8 (mini gastrin), pentagastrin, tetragastrin, and fragments, analogs, and derivatives thereof. Sequences for gastrins including big gastrin-34 (Bonato et al, 1986, Life Science 39:959) and small gastrin-17 (Bentley et al (1966) Nature 209:583) are known in the art, and some are shown in SEQ ID NOs. 1 to 9. In particular, sequences for gastrins include gastrin 71 of SEQ ID NO. 5 (amino acid residues 22 to 92), gastrin 52 of SEQ ID NO. 6, gastrin 34 (big gastrin) of SEQ ID NOs. 1 or 2, gastrin 17 (little gastrin) of SEQ ID NO. 3 or 4, gastrin 14 of SEQ ID NO. 7, and gastrin 6 of SEQ ID NO. 8 or 9. Gastrin-34 is essentially an extension of an amino acid sequence at the N-terminal end of gastrin-17. Big gastrin is cleaved in vivo to release gastrin-17. Glp at the N-terminal end of a gastrin is pyroglutamate, which is a naturally cyclized form of glutamate. In various embodiments, where cysteine or lysine is added to a terminus of gastrin having a pyroglutamate, the pyroglutamate is replaced with a glutamate, or the pyroglutamate is deleted. A gastrin 34 or gastrin-17 may be used in the invention where there is a methionine or a leucine at position 15, as shown in SEQ ID NOs: 1-4 herein. In some aspects a gastrin does not comprise a pyroglutamate.

Examples of gastrin compounds that may be used in the present invention include the compounds disclosed in U.S. Pat. No. 6,288,301. In some applications of the invention, a gastrin compound may be selected that is a peptide or non-peptide agonist or partial agonist of the gastrin receptor such as A71378 (Lin et al., Am. J. Physiol. 258 (4 Pt 1): G648, 1990).

In some applications of the invention, a gastrin compound may be selected that is a gastrin/CCK_{B} receptor ligand including but not limited to cholecystokinin (CCK) such as CCK 58, CCK 33, CCK 22, CCK 12 and CCK 8; and the like, or a cholecytokinin agonist.

A “gastrin compound” includes a modified form of a gastrin, including but not limited to a modified form of gastrin 71 [SEQ ID NO. 5, amino acid residues 22 to 92], gastrin 52 [SEQ ID NO. 6], gastrin 34 (big gastrin) [SEQ ID NO. 1 or 2], gastrin 17 (little gastrin) [SEQ ID NO. 3 or 4], gastrin 14 [SEQ ID NO. 7], gastrin 8, gastrin 6 [SEQ ID NO. 8 or 9], pentagastrin, and tetragastrin. A modified gastrin preferably comprises TrpMetAspPhe-NH₂ [SEQ ID NO. 13] or TrpLeuAspPhe-NH₂ [SEQ ID NO. 14].

In aspects of the invention a modified gastrin comprises at least amino acids 1-34, 18-34 or 29-34 of SEQ ID NO. 1 or 2, or amino acids 1-17, 2-17, 12-17, or 14-17 of SEQ ID NO. 3 or 4.

A gastrin compound used in aspects of the methods and compositions of the invention may comprise gastrin 17 and analogs and derivatives thereof. In particular aspects, the gastrin compound is synthetic human gastrin 1 having 17 amino acid residues with a Leu residue at amino acid position 15 [SEQ ID NO. 4].

A gastrin compound used in the methods and compositions of the invention may comprise gastrin 34 and analogs and derivatives thereof. In particular aspects, the gastrin compound is a synthetic human gastrin 34 with methionine or leucine at position 32 [SEQ ID NO. 1 or 2].

Modified gastrin compounds for use in the present invention comprise the modified gastrin compounds described in PCT/CA03/01778, U.S. Ser. No. 10/719,450 and U.S. Application Ser. No. 60/519,933 incorporated in their entirety by reference.

In particular, a modified gastrin can be a gastrin derivative or analogue comprising a minimal sequence of 6 amino acids (from the C-terminal end) of a gastrin, in particular amino acid residues 1 to 34, 18 to 34 or 29-34 of SEQ ID NO: 1 or 2, or amino acid residues 1-17, 2-17, 12-17, or 14-17 of SEQ ID NO. 3 or 4, and comprising a reactive group capable of undergoing an addition reaction. Examples of reactive groups include without limitation thiols, alpha amino groups, epsilon amino groups, carboxyl groups or aromatic rings. A reactive group is generally capable of linking a gastrin sequence, directly or indirectly via a crosslinking agent and/or spacer region, to a carrier.

A reactive group may be introduced by adding or substituting an amino acid comprising a reactive group, for example by adding a cysteine or lysine. Therefore, a modified gastrin may comprise a gastrin sequence (e.g. gastrin-34 or gastrin 17) wherein at least one reactive amino acid (e.g. cysteine or lysine) is added or substituted. The addition of a reactive amino acid can be at a terminal region, in particular an N-terminal region.

A modified gastrin may also optionally comprise a spacer. A spacer can interact with a reactive group, for example, an amino acid comprising a reactive group. A spacer can be one or more amino acids, peptides, peptidomimetics, or small organic molecules. A spacer can comprise at least one amino acid, preferably at least two, three, four or five amino acids and in certain embodiments it is a sequence of several amino acids, including without limitation alanine or glycine. A spacer can comprise alternating amino acids (e.g. glycine and/or alanine), non-alternating amino acids, a random sequence or a particular sequence. By way of example, a spacer can be synthesized as part of, or may be chemically attached to an amino acid of a gastrin sequence.

A modified gastrin may optionally comprise a cross-linking agent. A cross-linking agent may comprise a homobifunctional or heterobifunctional portion for interaction directly or indirectly with a gastrin, spacer and/or a reactive group. A cross-linking agent may interact with a gastrin sequence or a spacer, or it may be added to a reactive group at the end (in particular N-terminus) of a modified gastrin.

A cross-linking agent can be any agent that can link a gastrin sequence and a carrier directly or via a spacer. Examples of homobifunctional crosslinking agents include without limitation amino group directed homobifunctional cross-linking reagents such as bisimidates (e.g. methyl acetimidate-HCl), bifunctional aryl halides (e.g. 1,5-dichloro-2,4-dinitrobenzene), bifunctional acylating agents (e.g. diisocyanates), bifunctional sulfonyl halides (e.g. phenol-2,4-disulfonyl-chloride), bifunctional acylazides (e.g. tartryl diazide), dialdehydes (e.g. glutaraldehyde), and diketones (e.g. 2,5-hexanedione). Examples of heterobifunctional crosslinkers include amino and sulfhydryl group directed bifunctional reagents (e.g. N-succinimidyl-3-(2-pyridyldithio propionate), carboxyl and either sulfhydryl or amino group directed bifunctional reagents (e.g. p-nitrophenyl diazoacetate), and carbonyl and sulfhydryl group directed bifunctional reagents (e.g. 1-(aminooxy)-4-[3-nitro-2-pyridyl)dithio)]butane).

A modified gastrin can optionally comprise a carrier which may be a polymer. A carrier may be a polymer of amino acids (proteins), sugars (polysaccharides), nucleosides, synthetic polymers and mixtures thereof. A protein carrier may be a protein found in the circulatory system. Examples of protein carriers found in the circulatory system, in particular the human circulatory system, include without limitation plasma components such as serum, purified serum proteins such as albumin (in particular human serum albumin), transferrin, or an immunoglobulin, red blood cell proteins such as glycophorin A and AE-1, sugar binding proteins such as a lectin, inactivated enzymes, phosphate and sulphate binding proteins, and lipid binding proteins. Examples of other suitable polymeric carriers include without limitation cellulose and derivatives thereof, starch and derivatives thereof, heparin and derivatives thereof, and synthetic polymers such as polyethylene glycol (PEG) and dextran, and derivatives thereof. Carriers may be attached to a gastrin or spacer by way of reactive groups on, or introduced to, the carrier, gastrin, and/or spacer. For example, carriers can be covalently attached to reactive groups (such as thiol groups, alpha and epsilon amino groups, carboxyl groups or aromatic groups) on a gastrin or spacer which may be present or added by chemical modification of the gastrin or spacer.

In certain aspects of the invention, a modified gastrin can comprise a gastrin of SEQ ID NOS 1, 2, 3, 4, 7, 8, or 9 and a carrier.

A group of modified gastrin compounds include compounds having an amino acid sequence comprising from the amino terminus Z-Yₘ-Xₙ-AA₁-AA₂-AA₃-AA₄-AA₅-AA₆, wherein AA₁ is Tyr or Phe, AA₂ is Gly, Ala, or Ser, AA₃ is Trp, Val, or Ile, AA₄ is Met or Leu, AA₅ is Asp or Glu, and AA₆ is Phe or Tyr and wherein AA₆ is optionally amidated; Z is a carrier, in particular a polymer and when the polymer is a protein Z is an amino acid sequence; Yₘ is an optional spacer region comprising m amino acid residues of a small neutral amino acid including but not limited to serine and alanine, and X is any consecutive portion of residues 1-28 (=n) of SEQ ID NO: 1 or 2 or 1-11 of SEQ ID. NO. 3 or 4 providing that the gastrin compound binds a gastrin/CCK_{B} receptor. Generally, m is 0 to about 20 residues. In an aspect Z is a protein, in particular a protein of the circulatory system, more particularly a serum protein, still more particularly albumin, most particularly human serum albumin.

In embodiments, X is one or more amino acid residues from position 18 to position 28 of SEQ ID NO: 1. Therefore, the gastrin compounds by virtue of the presence of X can have any of the gastrin sequences from positions 18-28, 19-28, 20-28, 21-28, etc. The gastrin compound optionally contains an amino acid spacer (Y) of length m, and m is 0 to about 20 residues.

In embodiments, X is one or more amino acid residues from position 1 to 11 or 2 to 11 of SEQ ID NO: 3 or 4. Therefore, the gastrin compounds by virtue of the presence of X can have any of the gastrin sequences from positions 2 to 11, 3 to 11, 4 to 11, 5 to 11, etc. The gastrin compound optionally contains an amino acid spacer (Y) of length m, and m is 0 to about 20 residues.

A gastrin compound includes a modified gastrin compound of the formula Xₙ-AA₁-AA₂-AA₃-AA₄-AA₅-AA₆, where there is no spacer (Y) and m is 0, which may further comprise a bifunctional cross-linking agent for interaction or linkage to a carrier Z, where Z further comprises a non-proteinaceous polymer such as dextran or PEG.

A modified gastrin compound particularly described herein may further comprise an amino terminal cysteine or lysine residue.

In some embodiments of modified gastrin compounds described herein, the gastrin component contains at least amino acid residues 29-34 of SEQ ID NO: 1 or 2, and is associated with a polymer, a lipid or a carbohydrate. The polymer may be a synthetic or naturally occurring polymer. The term polymer includes a protein polymer of amino acids, and is not limited to a synthetic polymer. The polymer may be a polyethylene glycol (PEG) or a dextran. A modified gastrin compound can be based on SEQ ID NO: 1 or 2 or “big” gastrin-34 and have a residue at position 32 which is a methionine or a leucine, respectively.

Another preferred modified gastrin compound comprises a structure C-Yₘ-X, wherein C is Cys or Lys, Yₘ is an optional spacer region comprising m amino acid residues of a small neutral amino acid, and X is at least six amino acid residues comprising at least positions 12-17 of gastrin-17 (SEQ ID NO: 3 or 4) or at least positions 29-34 of gastrin-34 (SEQ ID NO: 1 or 2). This modified gastrin compound can further comprise a bifunctional cross-linking agent wherein one reactive portion of the cross-linking agent is covalently linked to C, and the other reactive portion is covalently linked to a polymer or protein.

In a particular aspect of the invention AA₁-AA₂-AA₃-AA₄-AA₅-AA₆ in a modified gastrin compound is Tyr-Gly-Trp-Met-Asp-Phe [SEQ ID NO. 10] or Tyr-Gly-Trp-Leu-Asp-Phe [SEQ ID NO.11].

In a further aspect of the invention, a gastrin compound used in the methods and compositions of the invention is gastrin 34 or gastrin 17 or portions thereof, directly or indirectly interacting or associated with a serum protein, in particular albumin or an immunoglobulin, more particularly human serum album.

In aspects of the invention, a gastrin compound comprises synthetic human gastrin 34 having 2-34 amino acid residues of SEQ ID NO. 1 or 2, and optionally an N-terminal cysteine and/or a carrier; synthetic human gastrin having 1-17 amino acid residues with a Leu residue at amino acid position 15 [SEQ ID NO. 4] and optionally an N-terminal cysteine residue; and a synthetic human gastrin having amino acid residues 2 to 17 or 5-17 of SEQ ID NO. 3 or 4, optionally with an N-terminal cysteine residue and/or a carrier (e.g. PEG or human serum albumin) linked via a spacer [e.g. Gly-Ala-Gly-Ala-Gly-Ala-Gly-Ala-Gly-Ala i.e. (GA)₅] [SEQ ID NO. 12], in particular, a synthetic human gastrin having amino acid residues 2 to 17 or 5-17 of SEQ ID NO. 3 or 4, with a human serum albumin (HSA) polymer linked via a Gly-Ala-Gly-Ala-Gly-Ala-Gly-Ala-Gly-Ala [i.e., (GA)₅] spacer, and optionally an N-terminal cysteine residue.

In particular aspects of the invention the gastrin compound is a leucine substituted gastrin 17 of SEQ ID NO. 4. Such a gastrin compound may also be characterized by one or more of the following properties: isoelectric point of about 3.4; purity of at least about 80%, 85%, 90%, 92%, 93%, 94%, 95%, 96%, 97%, 98%, or 99%, and/or a molecular mass of about 2080.2±2 Da.

Gastrin compounds may be synthesized by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart). The synthesis may be performed using manual procedures or by automation. Automated synthesis may be carried out, for example, using an Applied Biosystems 431A peptide synthesizer (Perkin Elmer). Gastrin compounds may also be obtained from commercial sources. For example, synthetic human gastrin 17 with methionine or leucine at position 15 are available from Bachem AG, Bubendorf, (Switzerland), and from Research Plus Inc (New Jersey, USA).

An “immunosuppressive agent,” refers to a compound or composition that induces immunosuppression, i.e., it prevents or interferes with the development of an immunologic response. Examples of immunosuppressive agents include, but are not limited to, Sandimmune®, or SangCya® (cyclosporines), Prograf®, Protopic® (tacrolimus); Rapamune®, Neoral® (sirolimus); FTY720; Certican® (everolimus, rapamycin derivative); Campath®-1H (alemtuzumab, anti-CD52 antibody); Rituxan (rituximab, anti-CD20 antibody); OKT4; LEA29Y (BMS-224818, CTLA41g); indolyl-ASC (32-indole ether derivatives of tacrolimus and ascomycin); Imuran® (azathioprine); Atgam® (antithymocyte/globuline); Orthoclone® (OKT3; muromonab-CD3); Cellcept® (mycophenolate mofetil); Zenapax® (daclizumab), Copaxone® (glatiramer acetate), Spanidin® (15-deoxyspergualin); Cytoxan®, Procytox® and Neosar® (cyclophosphamides), Purinethol®, Amevive®, Remicade®, interferon, Rheumatrex®, Trexall®, Novantrone®, Deltasone®, Simulect®, Thymoglobulin®, Xanelim®, Zenapax®, Allotrap®, Leustatin® (cladribine), prednisolone and other corticosteroids, malononitrilamides (MNAs), Arava® (leflunomide, FK778, FK779)), and 15-deoxyspergualin (DSG). Immunosuppressive agents may be produced by methods known in the art and they may be administered at therapeutically effective doses known in the art for the agents.

Specific examples of immunosuppressive agents include cyclosporine, tacrolimus, and sirolimus [ee, e.g., Khanna (2000) Transplantation, 70(4): 690-694; Khanna et al. (1999) Transplantation, 67(7): S84; and Khanna et al. (1999) Transplantation, 67(6):882-889]

An “insulin sensitivity enhancer” or “insulin resistance deblocker” refers to a substance that restores impaired insulin receptor function to deblock insulin resistance thereby enhancing insulin sensitivity. Exemplary insulin sensitivity enhancers are pioglitazone [Fujita et al., Diabetes, 32, 804-810, 1983, JP-A S55 (1980)-22636 (EP-A 0008203), JP-A S61 (1986)-267580 (EP-A 193256)], CS-045, PPAR(α) antagonists (fibrates), rexinoids, protein tyrosine kinase inhibitors β₃ adrenergic receptor antagonists, thiazolidinediones (TZDs) including thiazolidinedione derivatives and substituted thiazolidinedione derivatives which may optionally be used in combination with insulin [JP-A H4 (1992)-66579, JP-A H4 (1992)-69383, JP-A H5 (1993)-202042]. Insulin sensitivity enhancers may be produced by methods known in the art and they may be administered at therapeutically effective doses known in the art for the compounds.

In particular aspects of the invention insulin sensitivity enhancers include 5-[[3,4-dihydro-2-(phenylmethyl)-2H-1-benzopyran-6-yl]methyl]-2,4-thiazolidinedione (generic name: englitazone) or its sodium salt; 5-[[4-[3-(5-methyl-2-phenyl-4-oxazolyl)-1-oxopropyl]phenyl]methyl]-2,4-thiazalidinedione (generic name: darglitazone/CP-86325) or its sodium salt; 5-[2-(5-methyl-2-phenyl-4oxazolylmethyl)benzofuran-5-ylmethyl]-2,4-oxazolidinedione (CP-92768); 5-(2-naphthalenylsulfonyl)-2,4-thiazolidinedione (AY-31637); 4-[(2-naphthalenyl)methyl]-3H-1,2,3,5-oxathiadiazol-2-oxide (AY-30711); and 5-[[4-[2-(methyl-2-pyridylamino)ethoxy]phenyl]-methyl]-2,4-thiazolidinedione (BRL-49653). Certain thiazolidinedione insulin sensitizers are also disclosed in European Patent Applications, Publication Numbers: 0008203, 0139421, 0032128, 0428312, 0489663, 0155845, 0257781, 0208420, 0177353, 0319189, 0332331, 0332332, 0528734, 0508740; International Patent Application, Publication Numbers 92/18501, 93/02079, 93/22445 and U.S. Pat. Nos. 5,104,888 and 5,478,852.

In particular aspects of the invention an insulin sensitivity enhancer is a thiazolidinedione insulin sensitizer, in particular a thiazolidinedione insulin sensitizer including compounds comprising a 2,4-thiazolidinedione moiety. In embodiments of the invention the insulin sensitizer is a thiazolidinedione insulin sensitizer including without limitation (+)-5-[[4-[(3,4-dihydro-6-hydroxy-2,5,7,8-tetramethyl-2H-1-benzopyran-2-yl)methoxy]phenyl]methyl]-2,4-thiazolidine dione (or troglitazone), 5-[4-[(1-methylcyclohexyl)methoxy]benzyl]thiazolidine-2,4-dione (or ciglitazone), 5-[4-[2-(5-ethylpyridin-2-yl)ethoxy]benzyl]thiazolidine-2,4-dione (or pioglitazone) or 5-[(2-benzyl-2,3-dihydrobenzopyran)-5-ylmethyl)thiazolidine-2,4-dione (or englitazone), more particularly pioglitazone and rosiglitazone (Actos® and Avandia®)

Acyclic insulin sensitizers which have insulin sensitizer activity may also be utilized in the present invention. These sensitizers are illustrated in International Patent Applications, Publication Numbers WO93/21166 and WO94/01420, and in U.S. Pat. No. 5,232,945 and International Applications Nos. WO92/03425 and WO91/19702. Other exemplary insulin sensitizers are those disclosed in European Patent Application, Publication Number 0533933, Japanese Patent Application Publication Number 05271204 and U.S. Pat. No. 5,264,451.

A “glucose lowering agent” refers to a substance having one or more of the following actions: stimulates anaerobic glycolysis, increases the sensitivity to insulin in the peripheral tissues, inhibits glucose absorption from the intestine, suppresses hepatic gluconeogenesis, and inhibits fatty acid oxidation. Glucose lowering agents may be produced by methods known in the art and they may be administered at therapeutically effective doses known in the art for the compounds.

Glucose lowering agents that may be used in the present invention include biguanide compounds, thiazolidinediones, and alpha-glucosidase inhibitors. Biguanide compounds include but are not limited to N-dimethylbiguanides, substituted or otherwise, and for example metformin, but also other pharmaceutical compounds, for example buformin or fenformin, or a salt thereof with a therapeutically compatible mineral acid or organic acid. In aspects of the invention, a glucose lowering agent is metformin hydrochloride. Metformin is commercially available in 500 mg, 850 mg and 1000 mg tablets under the GLUCOPHAGE® trade name from Bristol Meyers Squibb. Metformin hydrochloride may be administered in humans at an initial daily dose of from 500 mg to about 800 mg and increased, as needed, to a maximum daily dosage of 2550 mg.

Alpha-glucosidase inhibitors, including without limitation acarbose (Precose®) and miglitol (Glycet®), inhibit α-glucosidase enzymes resulting in the reduction of glucose concentrations in the blood.

An “insulin secretagogue” is a compound which promotes increased secretion of insulin by the pancreatic beta cells. In general, an insulin secretagogue's action is initiated by binding to and closing a specific sulfonylurea receptor (an ATP-sensitive K⁺ channel) on pancreatic β-cells which decreases K⁺ influx, leading to depolarization of the membrane and activation of a voltage-dependent Ca²⁺ channel. Increased Ca²⁺ flux into the β-cell activates a cytoskeletal system that causes translocation of insulin to the cell surface and its extrusion by exocytosis. Insulin secretagogues include sulfonylureas, meglinitides, and amylin compounds.

A sulfonylurea useful for the methods and combinations of this invention may be a glyburide (DIABETA™), glipizide (GLUCOTROL™, GIBENESE™, MONODIAB™), glipizide (XL) (GLUCOTROL XL™), glimepiride (AMARYL™), glibenclamide (Daonil, Euglucon), gliclazide (Diamicron), gliquidone (Glurenorm), glibormuride, glisoxepide, glisentide, glisolamide, glyclopyamide, glycylamide, chlorpropamide (DIABINESE™), carbutamide, acetohexamide, tolbutamide, or tolazamide, or a pharmaceutically acceptable salt form of these agents. A combination of glyburide and metformin hydrochloride can also be commercially obtained under the GLUCOVANCE™ tradename (Bristol Meyers Squibb). A further suitable insulin secretagogue includes repaglinide. Each of these agents may be produced by methods known in the art. These agents may also be administered at the pharmaceutically or therapeutically effective dosages or amounts known in the art for these compounds such as those described in the Physician's Desk Reference 2001, 55 Edition, Copyright 2001, published by Medical Economics Company, Inc.

Meglinitides that can be used for the methods and combinations of this invention include repaglinide (Prandin®) and nateglinide (Starlix®).

An “amylin compound” refers to amylin and modulators thereof, in particular amylin agonists. The term “amylin” includes compounds such as those defined in U.S. Pat. No. 5,234,906 and U.S. Pat. No. 5,367,052. For example, it includes the human peptide hormone referred to as amylin and secreted from the beta cells of the pancreas, and species variations of it. The hormone is secreted along with insulin from the beta cells of the pancreas in response to a meal. [See for example, Rink et al., Trends in Pharmaceutical Sciences, 14:113-118 (1993); Gaeta and Rink, Med. Chem. Res., 3:483-490 (1994); and, Pittner et al., J. Cell. Biochem., 55S:19-28 (1994) for a review of the structure and biology of amylin.] The invention contemplates the use of a 37 amino acid amylin protein hormone that includes the sequences of SEQ ID NOs. 15 and 16, and in particular KCNTATCATQRLANFLVHSSNNFGAILSSTNVGSNTY (SEQ. ID. NOs. 15), and sequences that share substantial sequence similarity thereto.

An “amylin agonist” refers to a compound that binds to or otherwise directly or indirectly interacts with an amylin receptor or other receptor or receptors with which amylin itself may interact to elicit a biological response, a compound that mimics the function, activity, or effects of amylin, and/or peptide analogues of amylin useful as agonists of amylin. An amylin agonist may be a peptide or a non-peptide compound, and includes amylin agonist analogs. Amylin agonists useful in this invention include amylin agonist analogs disclosed and claimed in U.S. Pat. No. 5,686,411, U.S. Pat. No. 5,175,145, U.S. Pat. No. 6,136,784, U.S. Pat. No. 6,410,511, and US Application No. 2003/0092606 USA.)], ¹⁸Arg^{25,28,29} Pro-h-amylin, ¹⁸Arg^{25,28} Pro-h-amylin, des-¹Lys¹⁸Arg^{25,28}Pro-h-amylin, 18Arg^{25,28,29}Pro-h-amylin, des-¹Lys¹⁸Arg^{25,28,29}Pro-h-amylin, ^{25,28,29}Pro-h-amylin, des¹Lys^{25,28,29,}Pro-h-amylin, and ²⁵Pro²⁶Val^{25,28}Pro-h-amylin. Other amylin agonists include calcitonins and peptides or their equivalents having similar amino acid sequences to known calcitonins and having one or more of the known biological activities, in particular, the ability to increase circulating glucose concentration in humans. In an aspect the amylin agonist is Symlin®. Amylin compounds may be produced by methods known in the art and they may be administered at therapeutically effective doses known in the art for the compounds.

An “antiobesity agent” or “appetite regulating agent” refers to any substance that may be used in the treatment or prevention of obesity or to modulate appetite in a subject. The agents may be produced by methods known in the art and they may be administered at therapeutically effective doses known in the art for the compounds.

Examples of antiobesity and appetite regulating agents include without limitation anorectic agents such as bromocryptine, dexfenfluramine and the like, monoamine reuptake inhibitors such as sibutramine and the like, sympathomimetics such as phendimetrazine and the like, fatty acid uptake inhibitors such as orlistat or the like, thyromimetics such as triiodothyronine or the like, CART (cocaine amphetamine regulated transcript) agonists, catecholaminergic agents (e.g. diethylpropion, phentermine, phenylpropanolamine, mazindol), NPY (neuropeptide Y) antagonists, MC 4 (meianocortin 4) agonists, MC 3 (melanocortin 3) agonists, orexin antagonists, TNF (tumor necrosis factor) agonists, CRF (corticotropin releasing factor) agonists, CRF BP (corticotropin releasing factor binding protein) antagonists, urocortin agonists, melanin concentrating hormone antagonists, B₃ adrenergic receptor agonists, MSH (melanocyte-stimulating hormone) agonists or mimetics, INCH (melanocyte-concentrating hormone) antagonists, thyromimetic agents, dehydroepiandrosterone or an analog thereof, glucocorticoid receptor agonist or antagonist, ciliary neurotrophic factors, human agouti-related protein antagonists, CCK (cholecystokinin) agonists, monoamine re-uptake inhibitors, serotonin re-uptake inhibitors, serotonin and noradrenaline re-uptake inhibitors, mixed serotonin and noradrenergic compounds, 5HT (serotonin) agonists, dopamine agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR8 agonists, AGRP (Agouti related protein) inhibitors, opioid antagonists (such as naltrexone), PACAP (pituitary adenylyi cyclase activating peptide), cannabinoid receptor antagonists, and ciliary neurotrophic factor.

β₃-adrenergic receptor agonists include without limitation {4-[2-(2-[6 aminopyridin-3-yl]-2(R)-hydroxyethylamino)ethoxy]phenyl}acetic acid, {4-[2-(2-[6-aminopyridin-3-yl]-2(R)-hydroxyethylamino)ethoxy]phenyl} benzoic acid, {4-[2-(2-[6-aminopyridin-3-yl]-2(R)-hydroxyethylamino)ethoxy]phenyl}propionic acid, and {4-[2-(2-[6-aminopyridin-3-yl]-2(R)-hydroxyethylamino) ethoxy]phenoxy}acetic acid.

In an aspect of the invention the appetite regulating agent is an amphetamine-related appetite suppressant in particular phentermine or phentermine hydrochloride (see U.S. Pat. No. 2,408,345). In another aspect of the invention the appetite regulating agent is the gut hormone peptide W (PW) (Batterham R L, Bloom S R, Ann N Y Acad. Sci. (2003 June); 994:162-8), in particular the gut hormone fragment peptide YY3-36 peptide (W336).

In another aspect of the invention the antiobesity agent is leptin. In a further aspect of the invention, the antiobesity agent is dexamphetamine or amphetamine.

In a further aspect of the invention the antiobesity agent is a serotonin agonist, in particular fenfluramine or dexfenfluramine or dexfenfluramine hydrochloride, in particular fenfluramine and dexfenfluramine (see U.S. Pat. No. 3,198,834).

In a further aspect of the invention the antiobesity agent is a monamine reuptake inhibitor, in particular sibutramine or its hydrochloride salt (see U.S. Pat. No. 4,929,629) preferably in the form of Meridia™.

In a further aspect of the invention the antiobesity agent is a dopamine agonist, in particular bromocriptine (see U.S. Pat. Nos. 3,752,814 and 3,752,888

In a further aspect of the invention the antiobesity agent is a lipase inhibitor, in particular dexfenfluramine hydrochloride or orlistat (see U.S. Pat. No. 4,598,089 and U.S. Pat. No. 6,004,996; orlistat is commercially available under the trade name Xenical™).

In a further aspect of the invention the antiobesity agent is mazindol or phentermine. Phentermine is commercially available under the trade name Ionamin™.

In a further aspect of the invention the antiobesity agent is phen-fen, which is a combination of fenfluramine or its hydrochloride and phentermin.

In a further aspect of the invention the antiobesity agent is phendimetrazine (Bontril™, X-Trozine™) or its tartrate salt, diethylpropion (Tenuate™) or its hydrochloride salt, fluoxetine, sertaline or its hydrochloride salt, ephedrine or its sulphate salt, bupropion, topiramate, benzphetamine or its hydrochloride salt, phenylpropanolamine or its hydrochloride salt, or ecopipam.

In an embodiment of the invention, an antiobesity agent is selected from the group consisting of phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine and pharmaceutical salts thereof.

“Insulin” includes fast-, intermediate-, and long-acting insulins. In particular fast-acting insulins include regular insulins and prompt insulin zinc suspensions (semilente insulins); intermediate-acting insulins include isophane insulin suspensions (NPH insulins, isophane insulin) and the insulin zinc suspensions (lente insulins); and the long-acting insulins include protamine zinc insulin suspensions, and extended insulin zinc suspensions (ultralente insulins). The preparations may be available as either porcine or bovine insulins. The term includes recombinant human insulin available as regular and isophane insulins and as insulin zinc suspensions, as well as modified fast-acting insulin [Lys(B28), Pro(B29) human insulin analog, created by reversing the amino acids at positions 28 and 29 on the insulin B-chain].

Commercially available insulins include without limitation the fast-acting insulins available from Eli Lilly such as (a) Iletin® I (Regular); (b) Regular Iletin® II (Pork, 100 Units); (c) Regular Iletin® II (Concentrated, Pork, 500 Units); (d) Humalog® Injection (insulin lyspro, recombinant DNA origin); and (e) Humulin® R (regular insulin, recombinant DNA origin, 100 Units); the fast-acting insulins available from Novo Nordisk such as (a) Novolin® R (Regular, Human Insulin Injection (recombinant DNA origin) 100 Units); (b) Novolin® R PenFill 1.5 ml Cartridges (Regular, Human Insulin Injection (recombinant DNA origin) 100 Units); (c) Novolin® R Prefilled™ (Regular, Human Insulin Injection (recombinant DNA origin) in a 1.5 ml Prefilled Syringe, 100 units/ml); (d) Regular Purified Pork Insulin (100 Units/ml); and (e) Velosulin® BR (Buffered Regular Human Insulin Injection, 100 Units/ml); the intermediate-acting insulins available from Eli Lilly such as (a) Humulin® 50/50 (50% human insulin isophane suspension and 50% human insulin injection (rDNA origin), 100 Units); (b) Humuline® 70/30 (70% human insulin isophane suspension and 30% human insulin injection (rDNA origin), 100 Units); (c) Humulin® L (lente; human insulin (rDNA origin) zinc suspension, 100 Units)); (d) Humulin® N(NPH; human insulin (rDNA origin) isophane suspension, 100 Units); (e) Lente® Iletin® I, (insulin zinc suspension, beef-pork); (f) NPH Iletin® I (isophane insulin suspension, beef-pork); (g) Lente Iletin® II (insulin zinc suspension, purified pork); and (h) NPH Iletin® II, (isophane insulin suspension, purified pork); the intermediate-acting insulins available from Novo Nordisk such as (a) Novolin® L (Lente, Human Insulin Zinc Suspension (recombinant DNA origin), 100 Units/ml); (b) Novolin® N(NPH, Human Insulin Isophane Suspension (recombinant DNA origin), 100 Units/ml); (c) Novolin® N PenFill® 1.5 ml Cartridges; (d) Novolin® N Prefilled™ (NPH, Human Insulin Isophane Suspension (recombinant DNA origin) in a 1.5 ml Prefilled Syringe, 100 Units/ml); (e) Novolin® 70/30 (70% NPH, Human Insulin Isophane Suspension and 30% Regular, Human Insulin Injection (recombinant DNA origin), 100 Units/ml); (f) Novolin® 70/30 PenFill® 1.5 ml Cartridges; (g) Novolin® 70/30 Prefilled™ (70% NPH, Human Insulin Isophane Suspension and 30% Regular, Human Insulin Injection (recombinant DNA origin) in a 1.5 ml Prefilled Syringe, 100 Units/ml); (h) Lente Purified Pork Insulin (Zinc Suspension, USP 100 Units/ml); and (i) NPH Purified Pork Isophane Insulin Suspension (100 Units/ml); and long acting insulins such as Eli Lilly's Humulin® U (Ultralente® human insulin (recombinant DNA origin) extended zinc suspension).

The structure of agents identified by generic or trade names herein may be taken from the standard compendium “The Merck Index” or from databases such as PubMed (http://www.ncbi.nlm.nih.gov/entrez/query.fcgi), and patent databases (http://www.uspto.gov/patft/index.html; http://patentsl.ic.gc.ca/intro-e.html; http://register.epoline.org/espacenet/ep/en/srch-reg.htm). A person skilled in the art using these references is fully enabled to identify, manufacture and test the pharmaceutical indications and properties in standard test models, both in vitro and in vivo. The agents may also be administered at the pharmaceutically or therapeutically effective dosages or amounts known in the art for these compounds, such as those described in the Physician's Desk Reference 2001, 55 Edition, Copyright 2001, published by Medical Economics Company, Inc.

“Condition(s) and/or disease(s)” refers to one or more pathological symptoms or syndromes for which a gastrin compound provides a beneficial effect or therapeutic effect. The condition and/or disease may require reduction or normalization of blood glucose levels, increases in insulin levels, inhibition of gastric acid secretion, inhibition of apoptosis of β-cells, stimulation of proliferation or differentiation of β-cells, reduction of body weight and/or insulin dependence or usage.

Examples of conditions and/or diseases include but are not limited to dyslipidemia, hyperglycemia, severe hypoglycemic episodes, stroke, left ventricular hypertrophy, arrhythmia, bacteraemia, septicaemia, irritable bowel syndrome, functional dyspepsia, diabetes, catabolic changes after surgery, stress induced hyperglycemia, respiratory distress syndrome, gastric ulcers, myocardial infarction, impaired glucose tolerance, hypertension, chronic heart failure, fluid retentive states, metabolic syndrome and related diseases, disorders, or conditions, obesity, diabetes, diabetic complications as well as symptoms of other diseases in which tissue is damaged due to elevated glucose levels, including Alzheimer's Disease, Parkinson's Disease, and other age-related, tissue-degenerative diseases, as well as the artherogenic effects of elevated leptin, for example in patients with impaired glucose tolerance and obese non-diabetic patients.

The term, “diabetes” as used herein means any manifested symptoms of diabetes in any mammal including experimental animal models, and including human forms such as Type 1 and Type 2 diabetes, early stage diabetes, and a pre-diabetic condition characterized by mildly decreased insulin or mildly elevated blood glucose levels. A “pre-diabetic condition” describes a subject demonstrating a symptom in terms of insulin or glucose level, and/or demonstrating a susceptibility to diabetes or a related condition due to family history, genetic predisposition, or obesity in the case of Type 2 diabetes, and includes a subject who has previously had diabetes or a related disease, disorder, or condition and is subject to risk of recurrence.

Diseases, disorders, and conditions related to diabetes, in particular Type 2 diabetes, include without limitation, diabetic nephropathy, diabetic retinopathy and diabetic neuropathy, macular degeneration, coronary heart disease, myocardial infarction, diabetic cardiomyopathy, myocardial cell death, coronary artery diseases, peripheral arterial disease, stroke, limb ischemia, vascular restenosis, foot ulcerations, endothelial dysfunction and/or atherosclerosis.

In aspects of the invention, a condition and/or disease may be selected from the group consisting of (a) Type 1 or Type 2 diabetes mellitus and related diseases, disorders or conditions (including but not limited to diabetic nephropathy, diabetic retinopathy and diabetic neuropathy); (b) insulin resistance and syndrome X, obesity and related diseases, disorders or conditions (including but not limited to Insulin Resistance, Type 2 Diabetes Mellitus, Reproductive Disorders, Cardiovascular Disease, Pulmonary Disease, Gallstones and Fasting-induced cholecystitis, Cancers and Cutaneous Disease), Cushing's Syndrome, Hypothyroidism, Insulinoma, Craniopharyngioma and Other Disorders Involving the Hypothalamus; (c) congestive heart failure, left ventricular hypertrophy, survival post myocardial infarction (MI), coronary artery diseases, atherosclerosis, angina pectoris, thrombosis, (d) hypertension including hypertension in the elderly, familial dyslipidemichypertension and isolated systolic hypertension (ISH); increased collagen formation, fibrosis, and remodeling following hypertension (antiproliferative effect); impaired vascular compliance, stroke; all these diseases or conditions associated with or without hypertension, (e) renal failure, in particular chronic renal failure, glomerulosclerosis, nephropathy; (f) hypothyroidism; (g) endothelial dysfunction with or without hypertension, (h) hyperlipidemia, hyperlipoproteinemia, hypertryglyceridemia, and hypercholesterolemia, (i) macular degeneration, cataract, glaucoma, (j) skin and connective tissue disorders, and (k) restenosis after percutaneous transluminal angioplasty, and restenosis after coronary artery bypass surgery; and (l) peripheral vascular disease.

“Insulinotropic activity” refers to an ability of a substance to stimulate insulin secretion in response to elevated glucose levels to produce or increase glucose uptake by cells and decreased serum glucose or blood glucose levels. Methods known in the art can be employed to assay for insulinotropic activity. For example, in vitro and in vivo methods may be used that measure gastrin receptor binding activity, receptor activation (see the methods described in EP 619,322 to Gelfand et al and U.S. Pat. No. 5,120,712), and/or insulin or C-peptide levels. Compounds and compositions described herein have insulinotropic activity if islet cells secrete insulin in the presence of the compounds or compositions above background levels or levels in the absence of the compounds or compositions. A compound may be administered to an animal and the insulin concentration can be monitored over time.

“Islet neogenesis” means formation of new beta cells by differentiation, which may or may not have the characteristics of stem cells which have the ability to reproduce in an unlimited manner.

### DETAILED DESCRIPTION OF EMBODIMENTS OF THE INVENTION

The invention is related to compositions and methods that utilize at least one gastrin compound. In particular, the invention relates to compositions and methods for the prevention, intervention and/or treatment of a condition and/or disease discussed herein comprising at least one gastrin compound to provide one or more beneficial effects. In aspects of the invention, the compositions and methods of the invention provide enhanced beneficial effects, in particular sustained beneficial effects, relative to the absence of the compound(s).

In aspects of the invention, where the condition and/or disease is diabetes, beneficial effects, in particular sustained beneficial effects of a composition, treatment, or method of the invention may manifest as one or more of the following:
a) An increase in pancreatic insulin levels relative to the levels measured in the absence of the active compound after administration to a subject with symptoms of diabetes. Preferably the compound induces at least about a 0.05%, 0.1%, 0.5%, 1%, 2%, 5%, 10%, 15%, 20%, 30%, 33%, 35%, 40%, 45%, 50%, 70%, 75%, 80%, 90%, or 95%, more preferably at least about a 35%, 40%, 50%, 70%, 75%, 80%, 90%, or 95%, increase in pancreatic insulin levels in a subject.
b) A reduction of an absence of symptoms of islet inflammation after administration to a subject with symptoms of diabetes.
c) A decrease in blood glucose levels relative to the levels measured in the absence of the compound in subjects with symptoms of diabetes. Preferably, the compound induces at least about a 1%, 2%, 5%, 10%, 15%, 20%, 30%, 40%, 50%, 60%, 70%, 80%, or 90% decrease in blood glucose levels. Most preferably, the compound yields blood glucose levels about or close to the levels common in a normal subject.
d) An increase in C-peptide levels relative to the levels measured in the absence of the compound in subjects with symptoms of diabetes. Preferably, the compound induces at least about a 0.05%, 0.1%, 0.5%, 1%, 2%, 5%, 10%, 15%, 20%, 25%, 26%, 30%, 33%, 34%, 35%, 40%, 45%, 50%, 60%, 70%, 80%, or 90%, more preferably at least about a 15%, 20%, 25%, 30% or 35%, increase in C-peptide levels.
e) Maintenance of blood glucose levels at about normal for a prolonged period of time.
f) Maintenance of hemoglobin A1c or glycated hemoglobin at about normal levels for a prolonged period of time, in particular maintaining a % hemoglobin A1c at between 6-8%, more particularly at about 7%.
g) A reduction in destruction of beta-cells. Preferably the compound provides at least about a 1%, 2%, 5%, 10%, 15%, 20%, 30%, 40%, 50%, 60%, 70%, 80%, or 90% reduction in destruction of beta-cells.
h) An increase in beta-cell function. Preferably the compound induces at least about a 1%, 2%, 5%, 10%, 15%, 20%, 30%, 40%, 50%, 60%, 70%, 80%, or 90% increase in beta-cell function.
i) A decrease in insulin delivery or usage compared with the absence of the compound in diabetic subjects. Preferably, the compound provides at least about a 1%, 2%, 5%, 10%, 15%, 20%, 25%, 30%, 35%, 40%, 45%, 50%, 55%, 60%, 65%, 70%, 75%, 80%, 85%, 90%, 92%, 93%, 94%, 95%, 96%, 97%, 98%, 99%, 100%, 30-100%, 30-80%, or 35-75%, reduction in insulin delivery or usage.
j) A decrease in requirement for insulin injection/intake by at least about 5-99%, 5-95%, 10-98%, 10-95%, 10-90%, 10-80%, 10-70%, 10-60%, 10-50%, 10-40%, 10-30%, 10-20%, 20-100%, 20-75%, 30-100% 30-90%, 30-80%, 30-75%, 35-90%, 35-80%, or 35-75%.
k) A reduction, prevention, or slowing of the rate of disease progression in a subject with diabetes.
l) A reduction or prevention of the development of severe hyperglycemia and ketoacidosis with symptoms of diabetes.
m) An increase in survival in a subject with symptoms of diabetes.

In embodiments of the invention, beneficial effects or sustained beneficial effects comprise or consist essentially of two, three, four, five, six, seven, eight, nine, ten, eleven, twelve or thirteen of a) through m). In particular embodiments, beneficial effects or sustained beneficial effects comprise or consist essentially of a), b), and c); a), b), c), and d); a), b), c), d), and e); a), b), c), d), e), and f); a), b), c), d), e), f), and g); a), b), c), d), e), f), g), and h); a), b), c), d), e), f), g), h), and i); a), b), c), d), e), f), g), h), i) and j); a), d), and e); a), d), e), and h); a), d), e), h), and i); a), d), e), h), i), and j); a), b), c), d), e), h), i), and j); a), b), c), d), e), h), i), j), and k); b), c), d), and e); b), c), d), e), h), i), and j); b), h), i) and j); a) through e); a) through f); a) through g); a) through h); a) through i); a) through j); a) through k); a) through l); and a) through m).

In particular aspects of the invention, where the condition and/or disease is Type 2 diabetes, the beneficial effects of a treatment of the invention, particularly in subjects receiving a glucose lowering agent and/or insulin sensitivity enhancer, most particularly a metformin with or without a TZD, may manifest as one or more of the following:
a) A decrease in hemoglobin A1c (HbA1c) levels relative to the levels measured in the absence of the compound in subjects with Type 2 diabetes. Preferably, the compound induces at least about a 0.05%, 0.1%, 0.25%, 0.4%, 0.43%, 0.45%, 0.5%, 0.6%, 0.7%, 0.75%, 0.8%, 0.85%, 0.9%, 0.93%, 0.94%, 0.97%, 0.98%, 1%, 1.05%, 1.09%, 1.1%, 1.12%, 1.14%, 1.15%, 1.18%, 1.21%, 1.2%, 1.5%, 2%, 5%, 10%, 15%, 20%, 30%, 33%, 35%, 40%, 45%, or 50% decrease in HbA1c, preferably from a baseline. In aspects of the invention involving a gastrin-17(leu) [SEQ ID NO. 4], there is at least about a 0.1% to 1.5%, 0.1 to 2%, 0.1 to 1.5%, 0.4 to 1.5%, 0.5 to 1.5%, 0.95 to 1.5%, 0.1 to 1%, 0.5 to 1%, 0.6 to 1%, 0.7 to 1%, 0.8 to 1%, or 0.1 to 0.9% decrease in HbA1c levels more preferably at least about a 0.5 to 1.5% decrease in HbA1c levels from a baseline. In aspects of the invention, the baseline HbA1c levels are greater than or equal to about 5%, 6%, 7%, 8%, 9% or 10%.
b) A progressive decrease in glucose levels relative to the glucose levels determined in the absence of the compound in subjects with Type 2 diabetes.
c) A decrease or reduction in fasting glucose levels at 1, 2, 3, 4, 5 or 6 months post-treatment. In an aspect, the fasting glucose levels decrease by about 30-50 mg/dL, 35 to 50 mg/dl, or 40 to 50 mg/dL, more preferably by an average of about 40-50 or 45 mg/dL, in particular at 3 months post treatment. In another aspect, the fasting glucose levels decrease by about 30-50 mg/dL, 35 to 50 mg/dl, 35 to 40 mg/dL, or 35 to 50 mg/dL, more preferably by an average of about 35-50 or 35 mg/dL, in particular at 5 or 6 months post treatment.
d) A progressive increase in insulin levels relative to the insulin levels determined in the absence of the compound in subjects with Type 2 diabetes.
e) A progressive increase in the insulin to glucose ratio relative to the ratio determined in the absence of the compound in subjects with Type 2 diabetes, in particular at least about 1, 2, 3, 4, 5, 6, 7, 8, 9 or 12 months post-treatment.
f) A progressive decrease in fasting pro-insulin to insulin ratio relative to the ratio determined in the absence of the compound in subjects with Type 2 diabetes.
g) An increase in insulin levels in Type 2 diabetes patients that have an HBA1c decrease greater than 1%. Preferably, a gastrin-17(leu) [SEQ ID NO. 4) induces at least about a 5-99%, 5-95%, 10-99%, 10 to 95%, 20-99%, 20-95%, 30-99% 30-95%, 40-99%, 40-95%, 45-99%, 45-95%, 50-99%, 50-95%, 60-99%, 60-95%, 65-99%, 65-95%, 70-99%, 70-98%, 70-96%, 70-95%, 75-99%, 75-98%, 75-97%, 75-96%, 75-95%, 76-99%, 76-98%, 76-97%, 76-96%, 76-95%, 77-99%, 77-98%, 77-97%, 77-96%, 77-95%, 78-99%, 78-98%, 78-97%, 78-96%, 78-95%, 79-99%, 79-98%, 79-97%, 79-96%, 79-95%, 80-99%, 80-98%, 80-97%, 80-96%, 80-95%, preferably an 80-95% increase in insulin levels.
h) A progressive improvement in glucose tolerance at about months 1, 2, and/or 3 months post-treatment.
i) An increase in C-peptide levels in Type 2 diabetes patients that have an HBA1c decrease greater than 1%. Preferably, a gastrin-17(leu) [SEQ ID NO. 4) induces an about 1-90%, 1-80%, 1-70%, 1-60%, 1-50%, 1-45%, 1-40%, 1-35%, 5-50%, 5-45%, 5-40%, 5-35%, 10-50%, 10-45%, 10-40%, 10-35%, 15-50%, 15-45%, 15-40%, 15-35%, 16-50%, 16-45%, 16-40%, 16-39%, 16-38%, 16-37%, 16-36%, 16-35%, 17-50%, 17-45%, 17-40%, 17-39%, 17-38%, 17-37%, 17-36%, 17-35%, 18-50%, 18-45%, 18-40%, 18-39%, 18-38%, 18-37%, 18-36%, 18-35%, 19-50%, 19-45%, 19-40%, 19-39%, 19-38%, 19-37%, 19-36%, 19-35%, 20-50%, 20-45%, 20-40%, 20-39%, 20-38%, 20-37%, 20-36%, 20-35%, more preferably an about 20-35% increase in C-peptide levels.

In embodiments of the invention involving the treatment of Type 2 diabetes, beneficial effects or sustained beneficial effects comprise or consist essentially of two, three, four, five, six, seven, or eight of a) through i). In particular embodiments, beneficial effects or sustained beneficial effects comprise or consist essentially of a), b), and c); a), b), c), and d); a), b), c), d), and e); a), b), c), d), e), and f); a), b), c), d), e), f), and g); a), b), c), d), e), f), g), and h); a), b), c), d), e), f), g), and h); and a), b), c), d), e), g), h), and i).

In particular aspects of the invention, where the condition and/or disease is Type 1 diabetes, a beneficial effect of a treatment of the invention may manifest as a decrease in insulin delivery or usage compared with the absence of the compounds or for each compound alone in diabetic subjects. Preferably, the compounds provide at least about a 0.25%, 0.5%, 1%, 2%, 5%, 10%, 15%, 20%, 25%, 30%, 35%, 40%, 45%, 50%, 55%, 60%, 65%, 70%, 75%, 80%, 85%, 90%, 92%, 93%, 94%, 95%, 96%, 97%, 98%, 99%, 100%, 30-100%, 30-75%, 30-80%, 20-75%, preferably a 35 to 45% reduction in insulin delivery or usage and/or a decrease in HbA1c levels, preferably a decrease of at least about a 0.1%, 0.25%, 0.4%, 0.43%, 0.45%, 0.5%, 0.6%, 0.7%, 0.75%, 0.8%, 0.85%, 0.9%, 0.93%, 0.94%, 0.97%, 0.98%, 1%, 1.05%, 1.09%, 1.1%, 1.12%, 1.14%, 1.15%, 1.18%, 1.21%, 1.2%, 1.5%, 2%, 5%, 7%, or 10% in HbA1c levels, more preferably a 0.4-10%, 1-10%, 1-5%, 1-4%, 1-3%, 1-2%, 1-1.5%, 0.4-1.5%, 0.5%-1% or 0.5%-0.8% decrease.

In aspects of the invention, a gastrin compound comprises a sequence of one of SEQ ID NOs. 1 to 9 or modifications thereof.

In another aspect of the invention, a gastrin compound used in the methods and compositions of the invention is gastrin 34 and analogs and derivatives thereof. In a particular aspect, the gastrin compound is a synthetic human gastrin 34 with methionine or leucine at position 32 [SEQ ID NO. 1 or 2].

In particular aspects, a gastrin compound used in the methods or compositions of the invention is gastrin 17 and analogs and derivatives thereof. More particularly, the gastrin compound is a leucine substituted gastrin 17 of SEQ ID NO. 4. Such a gastrin compound may also be characterized by one or more of the following properties: isoelectric point of about 3.4; purity of at least about 80%, 85%, 90%, 92%, 93%, 94%, 95%, 96%, 97%, 98%, or 99%, and/or a molecular mass of about 2080.2±2 Da.

Pharmaceutical compositions of the invention can be selected that provide beneficial effects, in particular statistically significant beneficial effects or sustained beneficial effects. Beneficial effects in respect to a diabetic condition may be evidenced by one or more of the beneficial effects described herein. Pharmaceutical compositions may be used for preventing and/or treating a condition and/or disease, in particular diabetes, more particularly Type 2 diabetes and most particularly treating Type 2 diabetes in subjects receiving a glucose lowering agent and/or an insulin sensitivity enhancer (e.g. metformin with or without a TZD).

In an aspect, a pharmaceutical composition is provided comprising at least one gastrin compound. In a particular pharmaceutical composition, the gastrin compound is a gastrin of any one of SEQ ID NOs. 1 to 9 or modifications thereof.

In another aspect, a pharmaceutical composition, in particular with a beneficial effect(s), in particular statistically significant beneficial effect(s) or sustained beneficial effect(s), is provided comprising a gastrin compound of any one of SEQ ID NOs. 1 to 9 or modifications thereof, in particular gastrin-34(leu) [SEQ ID NO. 2] or gastrin-17(leu) [SEQ ID NO. 4], or a free base, free acid, salt, hydrate, ester, amide, enantiomer, isomer, tautomer, polymorph, metabolite or prodrug thereof.

In another aspect, a pharmaceutical composition with beneficial effects, in particular statistically significant beneficial effects or sustained beneficial effects, is provided comprising gastrin-17(leu) [SEQ ID NO. 4], or a free base, free acid, salt, hydrate, ester, amide, enantiomer, isomer, tautomer, polymorph, metabolite or prodrug thereof.

In another aspect, a pharmaceutical composition with beneficial effects, in particular statistically significant beneficial effects or sustained beneficial effects, is provided comprising one or more of at least one gastrin of any one of SEQ ID NOs. 1 to 9, in particular gastrin-34(leu) [SEQ ID NO. 2] or gastrin-17(leu) [SEQ ID NO. 4], or a free base, free acid, salt, in particular pharmaceutically acceptable salt, hydrate, ester, amide, enantiomer, isomer, tautomer, polymorph, metabolite or prodrug thereof.

In another aspect, a pharmaceutical composition with beneficial effects, in particular statistically significant beneficial effects or sustained beneficial effects, is provided comprising gastrin-34(leu) [SEQ ID NO. 2] or gastrin-17(leu) [SEQ ID NO. 4], or a free base, free acid, salt, in particular pharmaceutically acceptable salt, hydrate, ester, amide, enantiomer, isomer, tautomer, polymorph, metabolite or prodrug thereof.

In a particular aspect, a pharmaceutical composition with statistically significant beneficial effects or sustained beneficial effects is provided comprising gastrin-17(leu) [SEQ ID NO. 4] characterized by one or more of the following properties: isoelectric point of about 3.4; purity of at least about 80%, 85%, 90%, 92%, 93%, 94%, 95%, 96%, 97%, 98%, or 99%, and/or a molecular mass of about 2080.2±2 Da.

The invention in particular aspects provides a pharmaceutical composition which has been adapted for administration to a subject to provide sustained beneficial effects (e.g. effects that improve progressively post-treatment) to treat a condition and/or disease, preferably diabetes. In an embodiment for the prevention and/or treatment of diabetes, the composition is in a form such that administration to a subject results in control of haemoglobin A1c (HbA1c) levels, fasting blood glucose levels, glucose levels and/or insulin levels. In a particular embodiment, the composition is in a form such that administration to a subject results in a decrease in HbA1c levels by at least about 0.1%, 0.25%, 0.4%, 0.43%, 0.45%, 0.5%, 0.6%, 0.7%, 0.75%, 0.8%, 0.85%, 0.9%, 0.93%, 0.94%, 0.97%, 0.98%, 1%, 1.05%, 1.09%, 1.1%, 1.12%, 1.14%, 1.15%, 1.18%, 1.21%, 1.2%, 1.5%, or 2%, an increase in insulin levels by at least about 0.05%, 5%, 10%, 25%, 50%, 70%, 75%, 80%, 90%, or 95%, and/or an increase C-peptide levels by at least about 0.05%, 1%, 5%, 10%, 15%, 20%, 25%, 26%, 30%, 34%, 35%, 40%, 50%, 60%, 70%, 80%, or 90%. Preferably the effects persist in the subject for a prolonged period of time after cessation of treatment (e.g. 1, 2, 3, 4, 5, 6, 7, 8, 9, 10, 11 or 12, or more months post-treatment). In aspect of the invention, a pharmaceutical composition is administered for about 1 to 4 weeks, 1 to 5 weeks, 2 to 4 weeks, 2 to 6 weeks, 2 to 8 weeks, or 2 to 12 weeks, and stopped for about 1 to 4 months, 1 to 5 months, 1 to 6 months, 1 to 7 months, 1 to 8 months, 1 to 9 months, 1 to 12 months, 3 to 12 months, 3 to 9 months, 3 to 6 months, 6 to 9 months, 6 to 12 months, 6 to 18 months, 6 to 24 months, 9 to 12 months, 12 to 18 months, in particular 3 to 6 months or 3 to 9 months, more particularly 6 months.

In aspects of the invention, a composition comprises at least one gastrin compound having greater sustained insulinotropic activity following treatment compared with the absence of the compound.

The invention provides methods for the prevention, treatment and/or intervention of a condition and/or disease in a subject comprising administering at least one gastrin compound, or a pharmaceutical composition of the invention to provide a beneficial effect, in particular a sustained beneficial effect. In aspects of the methods, the subject has diabetes, in particular Type 2 diabetes. In particular aspects of the methods, the subject has baseline HbA1c levels greater than about 5%, 6%, 7%, 8%, 9% or 10%, in particular 5%, 6%, or 7%. In particular aspects of the methods, the subject is receiving a glucose lowering agent, an insulin sensitivity enhancer, and/or an insulin, in particular a metformin with or without a thiazolidinedione (TZD).

In certain methods of the invention the following is administered to a subject: one or more of a gastrin compound of any one of SEQ ID NOs. 1 to 9 or modifications thereof, in particular gastrin-34(leu) [SEQ ID NO. 2] gastrin-17(leu) [SEQ ID NO. 4], or a free base, free acid, salt, hydrate, ester, amide, enantiomer, isomer, tautomer, polymorph, metabolite or prodrug thereof.

In certain other methods of the invention, one or more of a gastrin of any one of SEQ ID NOs. 1 to 9, in particular gastrin-34(leu) [SEQ ID NO. 2] or gastrin-17(leu) [SEQ ID NO. 4]; or a free base, free acid, salt, hydrate, ester, amide, enantiomer, isomer, tautomer, polymorph, metabolite or prodrug thereof, is administered.

In certain other methods of the invention, a gastrin of any one of SEQ ID NOs. 1 to 9, in particular gastrin-34(leu) [SEQ ID NO. 2] or gastrin-17(leu) [SEQ ID NO. 4], or a free base, free acid, salt, in particular pharmaceutically acceptable salt, hydrate, ester, amide, enantiomer, isomer, tautomer, polymorph, metabolite or prodrug thereof, is administered.

In certain other methods of the invention, gastrin-17(leu) [SEQ ID NO. 4] or a free base, free acid, salt, in particular pharmaceutically acceptable salt, hydrate, ester, amide, enantiomer, isomer, tautomer, polymorph, metabolite or prodrug thereof, are administered.

In an aspect, the invention provides a method for the prevention and/or intervention of a condition and/or disease discussed herein in a subject comprising administration of at least one gastrin compound. The compounds may be directly administered to a subject or contacted with cells (e.g. stem cells or progenitor cells) and administered to a subject.

The invention also provides a treatment for preventing and/or treating a condition and/or disease discussed herein in a subject comprising administering to the subject a therapeutically effective amount of at least one gastrin compound to provide beneficial effects. In an aspect the invention provides a treatment or intervention which provides sustained beneficial effects following treatment.

In particular, the invention provides a treatment for treating or preventing a condition and/or disease in a subject comprising administering to the subject a therapeutically effective amount of at least one gastrin compound to produce beneficial effects, preferably sustained beneficial effects.

The invention also relates to a method of treatment comprising administering a therapeutically effective amount of at least one gastrin compound which upon administration to a subject with symptoms of diabetes produces beneficial effects, preferably sustained beneficial effects, manifested as reduced HbA1c levels and glucose levels, increased C-peptide levels and/or increased insulin levels.

In an aspect of the invention therapeutically effective amounts of at least one gastrin compound are combined with a pharmaceutically acceptable carrier prior to administration to a subject. In an embodiment, therapeutically effective amounts of at least one gastrin compound are mixed with a pharmaceutically acceptable carrier at a physiologically acceptable pH.

In a further embodiment, the invention provides a method for preventing or treating Type 1 or Type 2 diabetes comprising administering a therapeutically effective amount of a composition of the invention, or administering at least one gastrin compound.

In a still further embodiment, the invention provides a method for ameliorating progression of disease or obtaining a less severe stage of disease in a person suffering from Type 2 diabetes comprising administering a therapeutically effective amount of a composition of the invention, or administering at least one gastrin compound.

The invention relates to a method of delaying the progression of impaired glucose tolerance or non-insulin requiring Type 2 diabetes to insulin requiring Type 2 diabetes comprising administering a therapeutically effective amount of a composition of the invention, or administering at least one gastrin compound.

In a still further embodiment, the invention provides a method for ameliorating progression of disease or obtaining a less severe stage of disease in a person suffering from Type 1 diabetes comprising administering a therapeutically effective amount of a composition of the invention, or administering at least one gastrin compound.

The invention also relates to a method of increasing the insulin synthesis capability of a subject comprising administering a therapeutically effective amount of a composition of the invention, or administering at least one gastrin compound.

The invention further relates to inducing islet neogenesis in a subject comprising contacting pancreatic islet precursor cells with at least one gastrin compound, or a composition of the invention in a sufficient amount to increase proliferation of pancreatic islet precursor cells in the subject thereby inducing islet neogenesis.

The invention contemplates a method of expanding a functional beta cell mass of pancreatic islet transplants in a diabetic patient, the method comprising administering to the patient a therapeutically effective amount of at least one gastrin compound or a composition of the invention.

In an aspect, the invention provides methods for treating diabetes mellitus in a patient in need thereof by administering at least one gastrin compound, in an amount sufficient to effect differentiation of the patient's pancreatic islet precursor cells to mature insulin-secreting cells and/or to stimulate insulin synthesis in existing islet cells. The compound(s) can be administered systemically or expressed in situ by host cells containing one or more nucleic acid construct in an expression vector wherein the nucleic acid construct comprises a coding sequence for at least one gastrin compound together with transcriptional and translational regulatory regions functional in pancreatic islet precursor cells. Pancreatic islet precursor cells may be characterized as cells originating from insulin-producing islets with substantial proliferative potential, and capable of doubling in number about every 60 hours. The pancreatic islet precursor cells may also be characterized as cells expressing one or more marker protein including CK19, CK7, Ck8, Ck18, nestin, carbonic anhydrase II, DU-PAN2, carbohydrate antigen 19-9 and mucin MUC1.

Methods of the invention may further comprise measuring or monitoring one or more of the following markers: blood glucose, serum glucose, blood glycosylated haemoglobin, pancreatic beta cell mass, serum insulin, pancreatic insulin levels, morphometrically determined beta cell mass, C-peptide, amount of insulin secreting cells, and glucose responsiveness of insulin secreting cells.

The invention also contemplates the use of at least one gastrin compound for the preparation of a medicament, in particular providing beneficial effects, preferably sustained beneficial effects, in treating a condition and/or disease in a subject. The invention also contemplates the use of at least one gastrin compound for providing beneficial effects, preferably sustained beneficial effects in treating a condition and/or disease in a subject. In an aspect, the invention relates to the use of a therapeutically effective amount of at least one gastrin compound for preparation of a medicament for providing beneficial effects, preferably sustained beneficial effects, in treating a condition and/or disease. In an aspect, the invention relates to the use of a therapeutically effective amount of at least one gastrin compound for providing beneficial effects, preferably sustained beneficial effects, in treating a condition and/or disease in a subject. In an embodiment the invention provides the use of at least one gastrin compound for the preparation of a medicament to reduce HbA1c levels, decrease glucose levels, increase C-peptide levels and/or increase insulin levels. In an embodiment the invention provides the use of at least one gastrin compound for reducing HbA1c levels, decreasing glucose levels, increasing C-peptide levels and/or increasing insulin levels. In a still further embodiment the invention provides the use of at least one gastrin compound for treatment of Type 1 or Type 2 diabetes, or for the preparation of a medicament for treatment of Type 1 or Type 2 diabetes. In particular aspects, the subject has baseline HbA1c levels greater than about 5%, 6%, 7%, 8% 9% or 10%, in particular 5%, 6%, or 7%. In particular aspects, the subject is receiving a glucose lowering agent, an insulin sensitivity enhancer, and/or insulin, in particular a metformin with or without a thiazolidinedione (TZD).

The invention additionally provides uses of a pharmaceutical composition of the invention in the preparation of medicaments to provide beneficial effects, preferably sustained beneficial effects, in the treatment of conditions and/or diseases. The invention additionally provides uses of a pharmaceutical composition of the invention to provide beneficial effects, preferably sustained beneficial effects, in the treatment of conditions and/or diseases.

The methods, treatments and uses of the invention can comprise administering therapeutically effective amounts of at least one gastrin compound that provides beneficial effects including increased C-peptide levels, increased insulin levels, decreased HBA1c levels and/or about normal or reduced blood glucose levels. In preferred aspects, the beneficial effects, including sustained beneficial effects, comprise a decrease in HBA1c levels, in particular by at least about 0.1%, 0.25%, 0.4%, 0.43%, 0.45%, 0.5%, 0.6%, 0.7%, 0.75%, 0.8%, 0.85%, 0.9%, 0.93%, 0.94%, 0.97%, 0.98%, 1%, 1.05%, 1.09%, 1.1%, 1.12%, 1.14%, 1.15%, 1.18%, 1.21%, 1.2%, 1.5%, or 2%; an increase in insulin levels by at least about 0.05%, 5%, 10%, 25%, 50%, 70%, 75%, 80%, 90%, or 95%; and/or, an increase C-peptide levels by at least about 0.05%, 1%, 5%, 10%, 15%, 20%, 25%, 26%, 30%, 34%, 35%, 40%, 50%, 60%, 70%, 80%, or 90%. In selected methods, treatments and uses providing beneficial effects, more particularly sustained beneficial effects, the gastrin compound is a gastrin analogue, in particular gastrin-17(leu) of SEQ ID NO. 4. In particular aspects, the subject has baseline HbA1c levels greater than about 5%, 6%, 7%, 8% 9% or 10%, in particular 5%, 6%, or 7%. In particular aspects, the subject is receiving a glucose lowering agent, an insulin sensitivity enhancer, and/or insulin, in particular a metformin with or without a thiazolidinedione (TZD).

Therapeutic efficacy and toxicity of compounds and compositions of the invention may be determined by standard pharmaceutical procedures in cell cultures or with experimental animals such as by calculating a statistical parameter such as the ED₅₀ (the dose that is therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

The compositions of the present invention or fractions thereof typically comprise suitable pharmaceutical diluents, excipients, vehicles, or carriers selected based on the intended form of administration, and consistent with conventional pharmaceutical practices. The carriers, vehicles etc. may be adapted to provide an additive, complementary, synergistically effective or therapeutically effective amount of the active compounds. Suitable pharmaceutical diluents, excipients, vehicles, and carriers are described in the standard text, Remington: The Science and Practice of Pharmacy, 21^{st} Edition. University of the Sciences in Philadelphia (Editor), Mack Publishing Company.

For oral administration in the form of a capsule or tablet, the active components can be combined with an oral, non-toxic pharmaceutically acceptable inert carrier such as lactose, starch, sucrose, methyl cellulose, magnesium stearate, glucose, calcium, sulfate, dicalcium phosphate, mannitol, sorbital, and the like. For oral administration in a liquid form, the drug components may be combined with any oral, non-toxic, pharmaceutically acceptable inert carrier such as ethanol, glycerol, water, and the like.

Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof.

In an aspect of the invention a pharmaceutical composition has a pH from about 7 to 10.

Formulations for parenteral administration of a composition of the invention may include aqueous solutions, syrups, aqueous or oil suspensions and emulsions with edible oil such as cottonseed oil, coconut oil, almond oil, or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

Compositions for parenteral administration may include sterile aqueous or non-aqueous solvents, such as water, isotonic saline, isotonic glucose solution, buffer solution, or other solvents conveniently used for parenteral administration of therapeutically active agents. A composition intended for parenteral administration may also include conventional additives such as stabilizers, buffers, or preservatives, e.g. antioxidants such as methylhydroxybenzoate or similar additives.

In an embodiment, a solid form pharmaceutical composition is provided (e.g. tablets, capsules, powdered, or pulverized form) comprising a crystalline or amorphous gastrin compound.

In another embodiment, the invention relates to a liquid drug formulation comprising pharmaceutically acceptable salts of a gastrin compound and to lyophilized drug formulations that can be reconstituted to provide suspensions that are stable and suitable for parenteral administration.

In a particular embodiment, the invention relates to an aqueous composition comprising pharmaceutically acceptable salts of a gastrin compound, and a solvent system which effects solubilization. The invention also provides a drug comprising an aqueous formulation of pharmaceutically acceptable salts of a gastrin compound, with at least one solubilizer.

A composition of the invention may be sterilized by, for example, filtration through a bacteria retaining filter, addition of sterilizing agents to the composition, irradiation of the composition, or heating the composition. Alternatively, the compounds and compositions of the present invention may be provided as sterile solid preparations e.g. lyophilized powder, which are readily dissolved in sterile solvent immediately prior to use. After pharmaceutical compositions have been prepared, they can be placed in an appropriate container and labelled for treatment of an indicated condition and/or disease. For administration of a composition of the invention, such labelling would include amount, frequency, and method of administration.

In addition to the formulations described herein, the compositions can also be formulated as a depot preparation. Such long acting formulations may be administered by implantation (for example, subcutaneously or intramuscularly) or by intramuscular injection. Thus, for example, the fractions may be formulated with suitable polymeric or hydrophobic materials (for example, as an emulsion in an acceptable oil), or ion exchange resins, or as sparingly soluble derivatives, for example, as a sparingly soluble salt. The compositions of the invention and components thereof may comprise soluble polymers as targetable drug carriers.

The compounds, compositions, and medicaments of the present invention can be administered by any means that produce contact of the active agent(s) with the agent's sites of action in the body of a subject or patient. Active ingredients can be administered simultaneously or sequentially, and in any order at different points in time, to provide the desired beneficial effects. Each active ingredient may be independently administered any effective number of times, including more than once, as may be indicated by a physician or veterinarian.

The compounds, compositions and medicaments can be formulated for sustained release, for delivery locally or systemically. It lies within the capability of a skilled physician or veterinarian to select a form and route of administration that optimizes the effects of the compositions and treatments of the present invention.

The compounds, compositions and medicaments may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms all utilizing dosage forms well known to those of ordinary skill in the pharmaceutical arts. The compounds, compositions and medicaments may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen.

A particular route of administration is parenteral administration, preferably peripheral parenteral administration. Parenteral administration is generally understood to refer to the injection of a dosage form into the body by a sterile syringe or some other mechanical device such as an infusion pump. Parenteral routes include intravenous, intramuscular, subcutaneous, and intraperitoneal routes of administration. For parenteral administration, the compounds described herein may be combined with distilled water at an appropriate pH.

The present invention includes combination treatments providing additive or synergistic activity, delivering an additive or synergistically effective amount, or an amount to provide a therapeutically effective amount of at least one gastrin compound, or a composition of the invention, and one or more therapeutic agents disclosed herein. Therefore, pharmaceutical compositions suitable for use in the present invention include compositions wherein the active ingredients are contained in an additive, complementary, synergistically effective amount or a therapeutically effective amount.

The dosage regimen of the invention will vary depending upon known factors such as the pharmacodynamic characteristics of the agents and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect. The effective amount of a drug required to prevent, counter, or arrest progression of a condition can be readily determined by an ordinarily skilled physician or veterinarian.

A composition, medicament, or treatment of the invention may comprise a unit dosage of at least one gastrin compound.

A pharmaceutical composition of the invention can comprise a therapeutically effective suboptimal dosage of at least one gastrin compound, that is more effective at decreasing or reducing glucose levels for a sustained period following treatment compared with other compounds.

In an aspect, a pharmaceutical composition or treatment is provided comprising at least one gastrin compound in doses that are equal to or at least about 1.1, 1.5, 2, 3, 4, 5, 6, 7, 8, 9, or 10 fold lower than the doses of each compound required to provide beneficial effects, preferably sustained beneficial effects, to treat a condition and/or disease.

In another aspect the invention provides a pharmaceutical composition or treatment comprising between 0.1 to 20, 0.1 to 30, 0.1 to 40, 0.1 to 50, 0.1 to 60, 1 to 20, 1 to 30, 1 to 40, 1 to 50, 1 to 60, 5 to 30, 5 to 35, 5 to 40, 5 to 50, 10 to 30, 10 to 35, 10 to 40, 10 to 50, to 60, 15 to 20, 15 to 30, 15 to 35, 15 to 40, 20 to 40, 20 to 50, 20 to 60, 25 to 30, 25 to 35, 25 to 40, 25 to 45, or 30 to 35 micrograms/kg once, twice or more per day, preferably once per day, of a gastrin compound. In aspects of the invention, the subject is a human, in particular a human having a weight of 60 or 70 kg.

In an aspect the invention provides a pharmaceutical composition or treatment comprising between about 0.05 to 6 mg and within that range 100 to 2000 μg, 100-3000 μg, 100-4000 μg, 100-5000 μg, 100-6000 μg, 200 to 2000 μg, 200-3000 μg, 200-4000 μg, 200-5000 μg, 200-6000 μg, 300 to 2000 μg, 300-3000 μg, 300-4000 μg, 300-5000 μg, 300-6000 μg, 400 to 2000 μg, 400-3000 μg, 400-4000 μg, 400-5000 μg, 400-6000 μg, 500 to 2000 μg, 500-3000 μg, 500-4000 μg, 500-5000 μg, or 500-6000 μg, 600 to 2000 μg, 600-3000 μg, 600-4000 μg, 600-5000 μg, 600-6000 μg, 700 to 2000 μg, 700-3000 μg, 700-4000 μg, 700-5000 μg, 700-6000 μg, 800 to 2000 μg, 800-3000 μg, 800-4000 μg, 800-5000 μg, 800-6000 μg, 900 to 2000 μg, 900-3000 μg, 900-4000 μg, 900-5000 μg, 900-6000 μg, 1000 to 2000 μg, 1000-3000 μg, 1000-4000 μg, 1000-5000 μg, 1000-6000 μg, 1200 to 2000 μg, 1200-3000 μg, 1200-4000 μg, 1200-5000 μg, 1200-6000 μg, 1400 to 2000 μg, 1400-3000 μg, 1400-4000 μg, 1400-5000 μg, 1400-6000 μg, 1600 to 2000 μg, 1600-3000 μg, 1600-4000 μg, 1600-5000 μg, 1600-6000 μg, 1800 to 2000 μg, 1800-3000 μg, 1800-4000 μg, 1800-5000 μg, 1800-6000 μg, 2000-3000 μg, 2000-4000 μg, 2000-5000 μg, 2000-6000 μg, 2500-3500 μg, 2500-5000 μg, 2500-6000 μg, 3000-4000 μg, 3000-4500 μg, 3000-5000 μg, 3000-6000 μg, 4000-5000 μg, 4000-6000 μg, 5000-6000 μg, or 5500-6000 μg of gastrin compound per single unit.

A composition of the invention or components thereof may be administered to a subject continuously for 1 week, 2 weeks, 2 to 3 weeks, 3 to 4 weeks, 2 to 4 weeks, 2 to 6 weeks, 2 to 7 weeks, 2 to 8 weeks, 2 to 9 weeks, 2 to 10 weeks, 2 weeks to 12 weeks, 2 weeks to 16 weeks, 2 weeks to 20 weeks, 2 weeks to 24 weeks, 2 weeks to 6 months, 2 weeks to 16 weeks, 2 weeks to 6 months, 2 weeks to 12 months, or periodically, preferably 2 to 4 weeks.

The present invention also includes compositions and treatments of the invention in combination with or administered in combination with one or more additional therapeutic agents including without limitation immunosuppressive agents and antidiabetic agents. In aspects of the invention, the additional therapeutic agents include without limitation insulin sensitivity enhancers, glucose lowering agents, insulin secretagogues, insulin, antiobesity agents and appetite regulating agents, antihypertensive agents, and agents for the prevention and/or treatment of complications resulting from or associated with a condition and/or disease, in particular diabetes and obesity, anti-nausea medications, anti-headache medications, and general medications that treat or prevent side effects.

The invention also provides a kit comprising at least one gastrin compound, or a pharmaceutical composition in kit form, in particular for treatment of a subject with a condition and/or disease, in particular diabetes, more particularly a diabetic subject having baseline HbA1c levels greater than about 5%, 6%, 7%, 8%, 9% or 10%, in particular 5%, 6%, or 7%. The invention also relates to a pharmaceutical kit comprising one bottle with a gastrin compound, in one box. A kit may comprise a package which houses a container which contains a composition of the invention or components thereof and also houses instructions for administering the composition to a subject.

In embodiments of the invention, a pharmaceutical pack or kit is provided comprising one or more containers filled with one or more of the ingredients of a pharmaceutical composition of the invention to provide a beneficial effect, in particular a sustained beneficial effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

In an aspect, the invention relates to a “kit-of-parts”, for example, the components to be combined according to the present invention can be dosed independently or by use of different fixed combinations with distinguished amounts of the components, i.e. simultaneously or at different time points. The parts of the kit can then be administered simultaneously or chronologically staggered, that is, at different time points and with equal or different time intervals for any part of the kit.

Parts of a kit may be administered simultaneously or chronologically staggered, i.e., at different points in time and with equal or different time intervals for any component of a kit. Time intervals can be selected such that the effect on the condition and/or disease in the combined use of the parts is larger than the effect that would be obtained by use of only any one of the components.

The invention further relates to a commercial package comprising at least one gastrin compound, and optionally an additional therapeutic agent, together with instructions for simultaneous, separate or sequential use.

In an aspect a commercial package comprising as active ingredients at least one gastrin compound is provided in the form of a separate unit, and optionally another therapeutic agent, together with instructions for its simultaneous, separate or sequential use, or any combination thereof, in the delay of progression or treatment of a condition and/or disease disclosed herein.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

### A Randomized, Double-Blind, Placebo-Controlled Phase IIa Clinical Trial to Evaluate the Safety, Tolerability, and Effects of Daily Doses of an EGF (E1) in Combination with a Gastrin (G1) in Patients with Type 1 Diabetes

### Study Design

The Phase IIa clinical studies for Type 1 diabetes were randomized, double-blind, placebo-controlled trials to evaluate the safety, tolerability, and efficacy of daily treatments of an EGF (E1) in combination with a gastrin (G1) for 28 days with a 6-month follow-up. A total of 20 patients with Type 1 diabetes were randomized on Day 1 of the Treatment Phase. Fifteen (15) patients were randomized to receive active study medication and 5 patients were randomized to receive vehicle control. The study had a 2 week baseline period, 4 week Treatment period (daily treatments), and 6 month (non investigational drug treatment period). After undergoing screening procedures the patients entered a 14 day baseline phase where baseline data was collected. During this period and throughout the study, patients remained on their insulin regimen and insulin intake and blood glucose levels were recorded daily through the use of a daily diary.

Patients that successfully completed the baseline Phase entered the treatment phase where they were randomized to receive either once daily sc injections of E1 plus G1, as separate injections or once daily sc injections of vehicle control (as separate injections to mimic active treatment). Patients received once daily doses in the morning after breakfast for a period of 28 days. Patients randomized to active treatment received treatment according to the following schedule: During the first week of treatment, patients received 0.3 μg/kg E1 and 30 μg/kg of G1. Five patients received 0.3 μg/kg E1 and 30 μg/kg of G1 for the duration of the treatment period. When tolerance to 0.3 μg/kg E1 and 30 μg/kg of G1 was acceptable, the patients received 0.5 μg/kg E1 and 30 μg/kg of G1 during the second week of treatment and for the duration of the treatment period (5 patients). Patients not tolerating the second dose level of 0.5 μg/kg E1 and 30 μg/kg of G1 were stepped down to 0.3 μg/kg E1 and 30 μg/kg of G1 and they remained on 0.3 μg/kg E1 for the duration of the treatment period (2 patients). One patient started at 0.7 μg/kg E1 and was stepped down to 0.3 μg/kg E1 and remained on 0.3 μg/kg E1 through the treatment period.

All patients were on insulin therapy to regulate their blood-glucose levels and continued on this therapy throughout the trial.

On Days 1 to 3 of the treatment phase patients received study medication in the morning and stayed in the clinic throughout the day and overnight. On Day 4 patients remained in the clinic for at least 2 hr after receiving study medication and were released if the treatment was deemed to be well tolerated. They returned to the clinic every morning for the next 3 days to receive study medication and remained in the clinic for at least 30 min. after each dose. On Day 8, if tolerance was acceptable the dose was increased. On Day 9 patients remained in the clinic for at least 4 hours after receiving study medication and they were released if the treatment was deemed to be well tolerated. Patients returned to the clinic every morning for the next 19 days to receive study medication and they remained in the clinic for at least 30 min. after each dose.

Upon completion of treatment, all patients continued in the follow-up phase for an additional 6 months. During the 6-months follow-up period, patients were instructed to continue to record their daily insulin intake and glucose levels in a daily diary and they returned for monthly clinic visits.

### Study Population

Twenty (20) Type 1 diabetes patients requiring insulin therapy, male or female, ages 18-40 years inclusive.

### Study Treatments

E1, Epidermal Growth Factor (EGF) analogue (EGF 1-51 glu⁵¹asn), 1 mg/mL
G1, Gastrin analogue [gastrin-17(leu) of SEQ ID NO. 4], 4 mg/mL
Vehicle Control—0.9% normal saline

### Endpoints

Blood glucose levels

Basal & Arg stimulated C-peptide levels

Hemoglobin A_{1C} (HbA_{1c} levels

Insulin usage

### Results

There were no serious adverse events or deaths noted during the study. Any reported side effects of treatment were manageable, transient and did not appear to pose any acute safety risk. The most common adverse events reported were nausea, diarrhea, headaches, and vomiting which were generally mild to moderate.

Data analysis was completed up to 3 months post-treatment with assessment time points at 1, 2, and 3 months post-treatment. Patients who completed both 28 days of treatment and 3 months post-treatment follow-up were included in the efficacy analysis. In the study, 17 of 20 patients completed the treatment phase of the study (13 of 15 treated with E1+G1, 4 of 5 placebo). Two treated patients withdrew from the study due to adverse events. Episodes of hypoglycaemia occurred with similar frequency in both the treated and placebo groups.

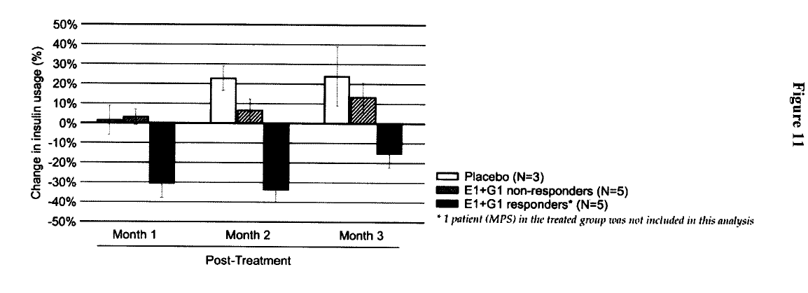

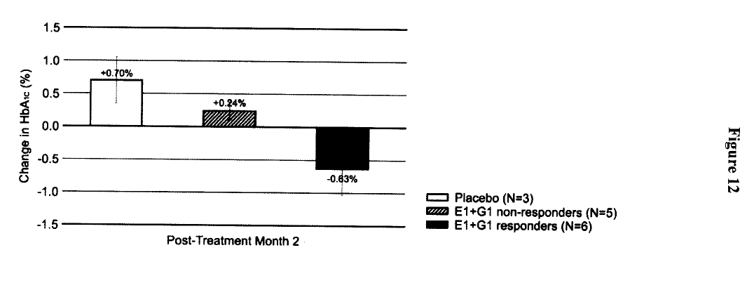

The analyses of changes in insulin usage and Hemoglobin A1c (HbA1c) levels are shown in FIGS. 11 and 12, respectively.

The treatment for 28 days showed positive data or trends in some patients with Type 1 diabetes. Approximately 50% of the patients showed an average of 37% maximal decrease in insulin use (ranging from 20% to 75% in months 1, 2 or 3 post-treatment). A single patient had no change in insulin use but showed a decrease in HbA1c levels from 6.7% in baseline to 5.5%, 4.8%, and 4.7% in months 1, 2 and 3 post-treatment, respectively. Type 1 diabetes patients on average showed a trend of better glycemic control (Ale) and used less insulin compared to patients receiving placebo.

In this Type 1 diabetes study, 6 of 11 (54%) patients responded to the therapy, either by decreasing their average daily insulin usage by more than 20% or reducing their HbA1c levels by 1.2 to 2%. There were no responders among the placebo group.

These data indicate that a combination EGF receptor ligand and gastrin compound therapy can be clinically beneficial to patients with Type 1 diabetes. These data also indicate that a therapy with gastrin alone may also be useful.

### Example 2

### A Randomized, Double-Blind, Placebo-Controlled Clinical Trial to Evaluate the Safety, Tolerability and Clinical Response of Repeated Subcutaneous Doses of E1 in Combination with G1 in Patients with Type 2 Diabetes

### Study Design

The study was a randomized 2:1 treatment to vehicle control, double-blind design. A total of 30 patients with Type 2 diabetes were randomized on Day 1 of the Treatment Phase. Twenty (20) patients were randomized to receive active study medication and 10 patients were randomized to receive vehicle control.

This study had a 2 week baseline period, a 4 week treatment period, and a 6 month follow-up period. After undergoing Screening procedures the patients entered a 14 day baseline phase where baseline data was collected. During the baseline period and throughout the study, patients remained on their current oral hypoglycemic therapy with Metformin, Thiazolidinedione or both Metformin and Thiazolidinedione and blood glucose levels were recorded daily through the use of a daily diary. Patients were asked to measure capillary blood glucose measurements daily before each meal and at bedtime. Once a week, patients performed a 7-point profile, which consisted of the usual pre-meal glucose measurement plus a 2 hour post-meal sample after breakfast, lunch and dinner and the bedtime sample.

After successful completion of the baseline phase, patients entered the treatment phase where they were randomized to receive either once daily sc injections of E1 plus G1, as separate injections or once daily sc injections of vehicle control (as 2 separate injections to mimic active treatment). Patients received once daily doses in the morning after breakfast for a period of 28 days. Patients randomized to active treatment received treatment according to the following schedule: During the first week of treatment, patients received 0.3 μg/kg E1 and 30 μg/kg of G1. If tolerance to 0.3 μg/kg E1 and 30 μg/kg of G1 was acceptable, the dose was escalated to 0.5 μg/kg E1 and 30 μg/kg of G1 during the second week of treatment and for the duration of the treatment period (8 patients). Two patients started at 0.3 μg/kg E1 and 30 μg/kg of G1 and did not escalate up to 0.5 μg/kg on Day 8; they remained on 0.3 μg/kg E1 through Day 28. Patients not tolerating the dose levels of 0.3 μg/kg or 0.5 μg/kg E1 were stepped-down, in a blinded fashion, to the lower dose, i.e. 0.3 μg/kg E1 or 0.2 μg/kg E1 (4 patients).

On Days 1 to 3 of the treatment phase patients received study medication in the morning and stayed in the clinic throughout the day and overnight. On Day 4 patients remained in the clinic for at least 2 hours after receiving study medication and if the treatment was deemed to be well tolerated the patients were released. Patients returned to the clinic every morning for the next 3 days to receive study medication and remained in the clinic for at least 30 min. after each dose. In the event that patients experienced unacceptable tolerability during this 30 min. period, the appropriate duration of clinic time was decided prior to patient release. On Day 8, if tolerance was acceptable and dose was increased, patients stayed in the clinic throughout the day and overnight. On Day 9 patients remained in the clinic for at least 4 hours after receiving study medication and if the treatment was deemed to be well tolerated the patients were released. In the event that patients experienced unacceptable tolerability during this 4 hour period, the appropriate duration of clinic time was decided prior to patient release. Patients returned to the clinic every morning for the remainder of the treatment period to receive study medication and they remained in the clinic for at least 30 min. after each dose.

Upon completion of treatment, all patients continued in the follow-up phase for an additional 6 months. During the 6-months follow-up period, patients were instructed to continue to record their daily Metformin and/or Thiazolidinedione intake and glucose levels in a daily diary and return for monthly clinic visits.

### Study Population

Thirty (30) Type 2 diabetes patients requiring Metformin and/or Thiazolidinedione therapy, male or female, ages 30-60 years inclusive.

### Study Treatments

E1, Epidermal Growth Factor (EGF) analogue (EGF 1-51 glu⁵¹asn), 1 mg/mL
G1, Gastrin analogue [gastrin-17(leu) of SEQ ID NO. 4], 4 mg/mL
Vehicle Control—0.9% normal saline

E1 and G1 solutions were supplied to the site as sterile frozen solutions in PBS. During the Treatment Phase, each patient received 2 daily sc injections; either 1 each of E1 and G1 or 2 injections of vehicle control.

### Endpoints

Hemoglobin A_{1C} (HbA_{1c}) levels

OGTT (glucose, insulin, proinsulin, C-peptide)

Blood glucose levels

Arg stimulated C-peptide levels

E1 and G1 antibodies

IA2 and GAD antibodies

### Results—3 Months Post Treatment

No serious adverse events or deaths were noted during the trial. Any reported side effects of treatment were manageable, transient and did not appear to pose any acute safety risk. The most common adverse events reported were nausea, diarrhea, headaches, and vomiting which were generally mild to moderate.

Data analysis was completed up to 3 months post-treatment with assessment time points at 1, 2, and 3 months post-treatment. Patients who completed both 28 days of treatment and 3 months post-treatment follow-up were included in the efficacy analysis. 23 of 30 patients completed the treatment phase of the study (14 of 20 treated with E1+G1, 9 of 10 placebo). Of the six patients that discontinued the treatment, four patients withdrew from the trial after experiencing adverse events and two patients withdrew consent in the trial because of patient protocol violations or not complying with study requirements. No episodes of hypoglycaemia were reported in Type 2 diabetes patients in the trial. In particular, there were no episodes of hypoglycaemia events (defined as symptomatic or asymptomatic glucose of <70 mg/dl).

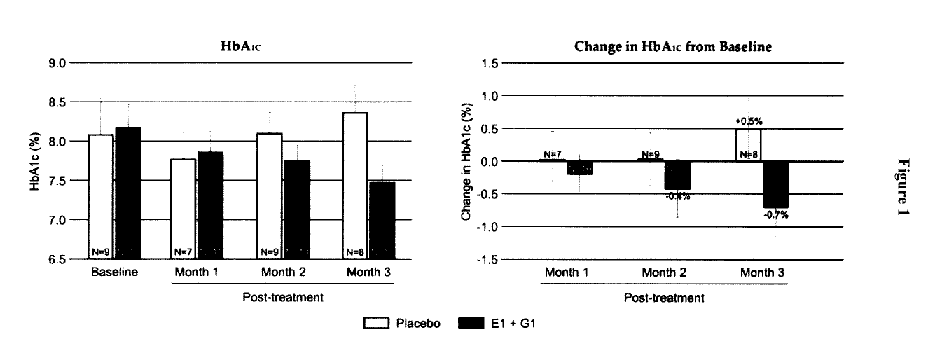

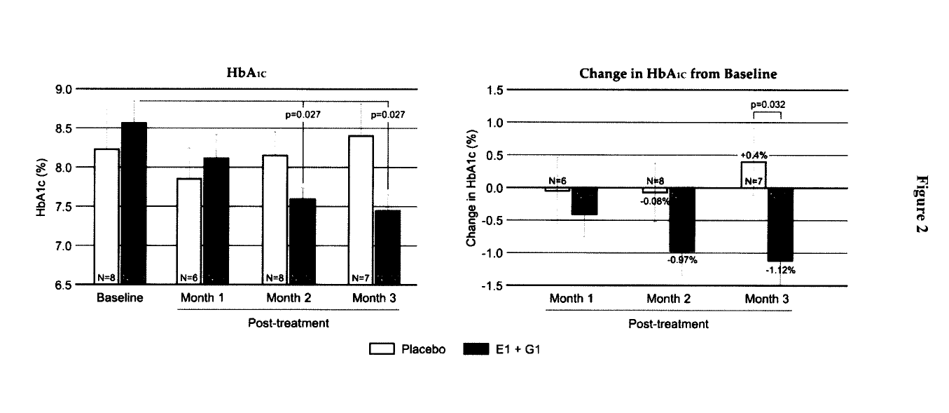

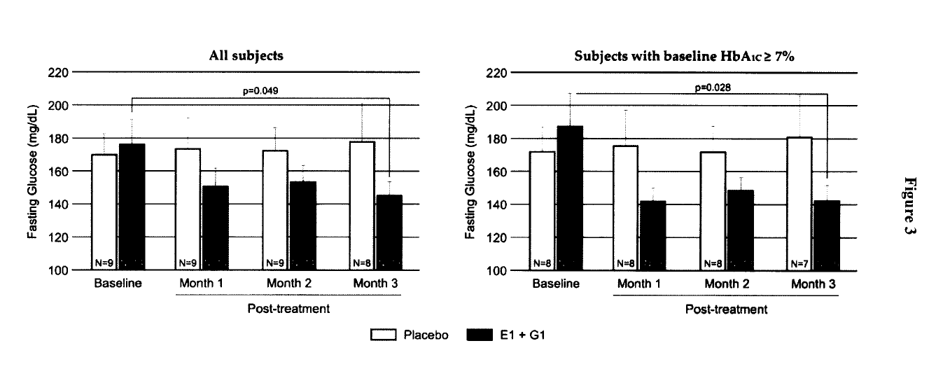

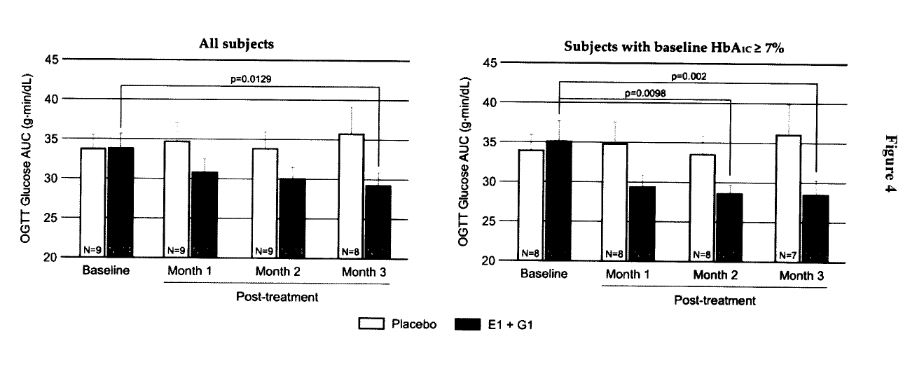

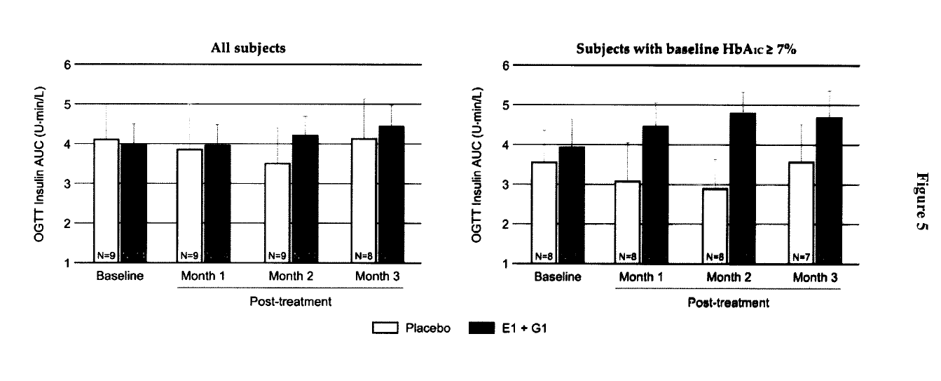

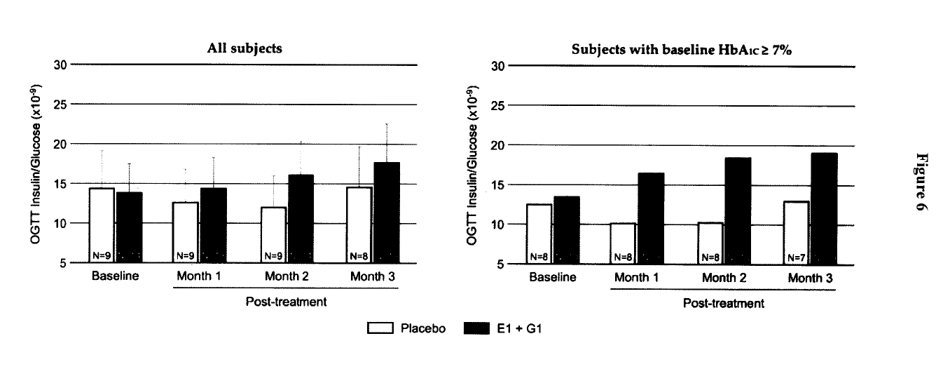

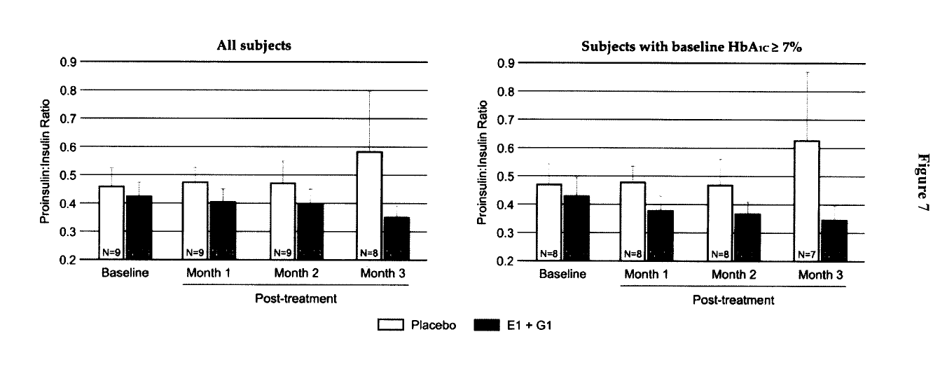

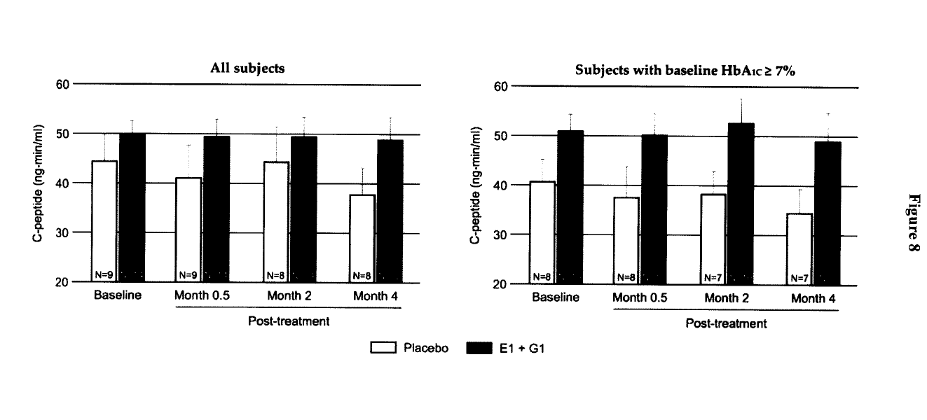

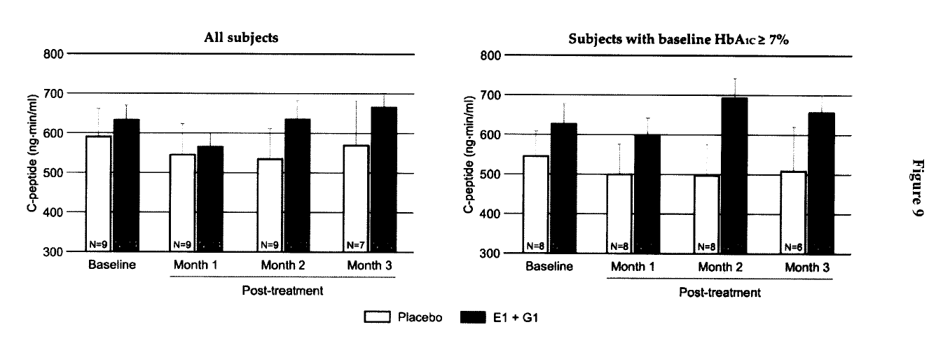

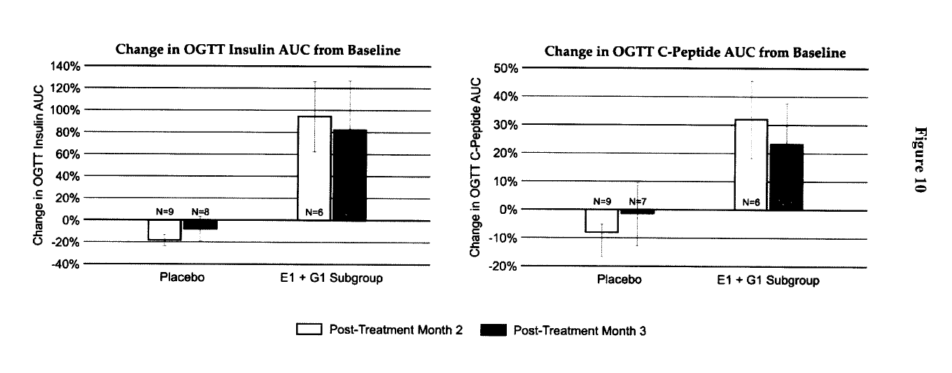

Upon entry into the trial, the Type 2 diabetes patients' Hemoglobin A1c (HbA1c) levels ranged between 6.8% and 10.9%, with a mean HbA1c level of 8.1%. The analyses of Hemoglobin A_{1C} (HbA_{1c}) levels, fasting glucose levels, glucose levels, insulin levels, proinsulin levels, insulin usage and C-peptide levels are shown in FIGS. 1 to 10.

The treatment for 28 days showed positive data or trends in most diabetes efficacy parameters examined in Type 2 diabetes patients with baseline HbA_{1c} levels greater than or equal to 7%. HbA1c levels decreased from baseline by an average of 0.97% (p=0.027) and 1.12% (p=0.027) in months 2 and 3 post-treatment, respectively, while HbA1c levels in placebo patients decreased by an average of 0.08% in month 2 post-treatment and increased by 0.4% in month 3 post-treatment. Fasting glucose levels were reduced with an average decrease of 45 mg/dL (p=0.0234) at 3 months post-treatment compared to an increase of 8 mg/dL in the placebo patients. Glucose tolerance improved in months 1, 2 (p=0.0137) and 3 (p=0.0134) post-treatment. Insulin levels showed a positive trend in months 1, 2 and 3 post-treatment. Insulin to glucose ratio showed a positive trend in months 1, 2, and 3 (p=0.07) post-treatment.

The HbA1c reductions shown in this trial were consistent with observed reductions in fasting blood glucose as well as improvements in glucose tolerance and increases in insulin levels as measured with an oral glucose tolerance test. These are area-under-the curve measurements which assess the parameter at several time points.

In particular as shown in the Figures, OGTT glucose AUC decreased progressively in months 1, 2 (p=0.0068) and 3 (p=0.0059) post-treatment. OGTT insulin AUC increased in months 1, 2 and 3 post-treatment (data did not show significance). OGTT insulin to glucose ratio increased progressively in months 1, 2 and 3 (p=0.044) post-treatment. Fasting pro-insulin to insulin ratio decreased progressively in months 1, 2 and 3 post-treatment (data did not show significance). OGTT C-peptide levels did not change significantly in the treated group, but did appear to be higher relative to placebo. OGTT insulin and C-peptide levels increased by 80-95% and 20-35% in months 2 and 3 post-treatment, respectively, in patients that had an HbA1C decrease greater than 1%.

Data from the Type 2 diabetes patients demonstrated that the treatment significantly lowered blood glucose levels for patients using metformin with/without thiazolidinediones (TZD). Type 2 diabetes patients showed improvements in multiple important measures of blood glucose control including HbA1c, fasting blood glucose, glucose levels, and insulin levels. The A1c levels (a measure of blood glucose control over time) decreased by an average of 0.97% (p=0.027) and 1.12% (p=0.027) in months 2 and 3 post-treatment, post-treatment, respectively in Type 2 diabetes patients with baseline HbA1c levels greater than or equal to 7%.

### Results—6 Months Post Treatment

No serious adverse events or deaths were noted during the trial. Any reported side effects of treatment were manageable, transient and did not appear to pose any acute safety risk. The most common adverse events reported were nausea, diarrhea, headaches, and vomiting which were generally mild to moderate.

Data analysis was completed up to 6 months post-treatment with assessment time points at 1, 2, 3, 4, 5 and 6 months post-treatment. Patients who completed both 28 days of treatment and 6 months post-treatment follow-up were included in the efficacy analysis. 23 of 30 patients completed the treatment phase of the study (14 of 20 treated with E1+G1, 9 of 10 placebo). Of the six patients that discontinued the treatment, four patients withdrew from the trial after experiencing adverse events and two patients withdrew consent in the trial because of patient protocol violations or not complying with study requirements. No episodes of hypoglycaemia were reported in Type 2 diabetes patients in the trial. In particular, there were no episodes of hypoglycaemia events (defined as symptomatic or asymptomatic glucose of <70 mg/dl).

Upon entry into the trial, the Type 2 diabetes patients' Hemoglobin A1c (HbA1c) levels ranged between 6.8% and 10.9%, with a mean HbA1c level of 8.1%.

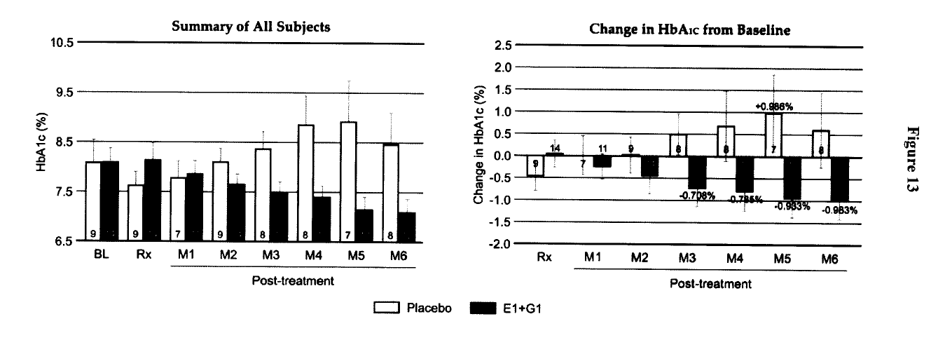

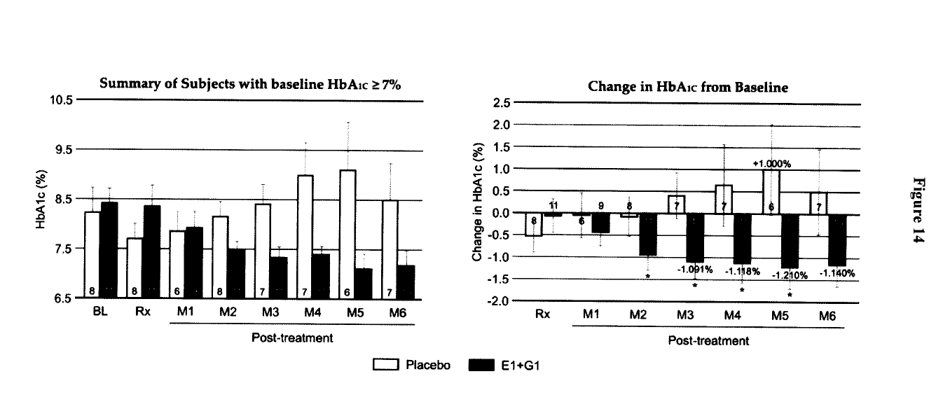

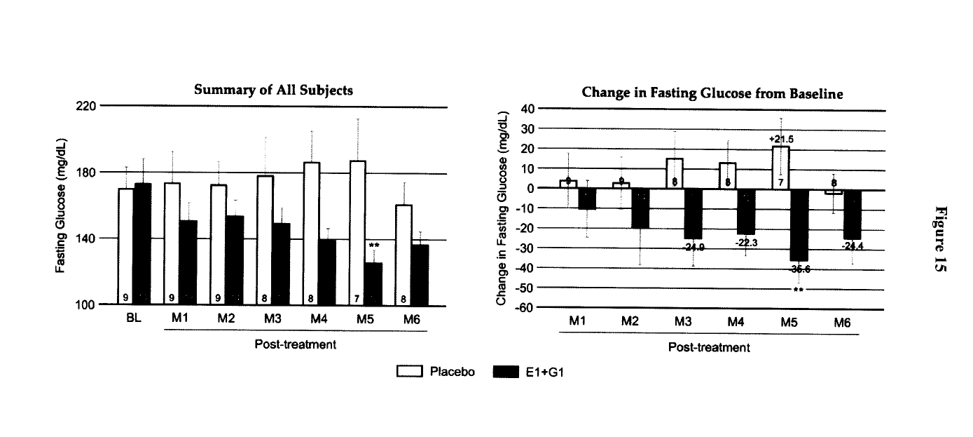

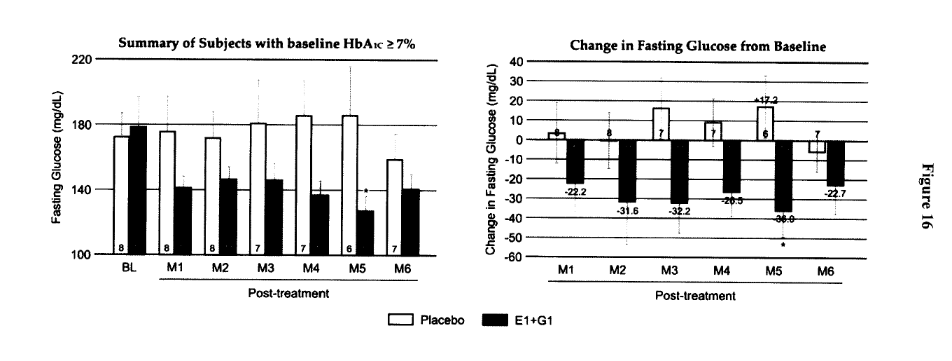

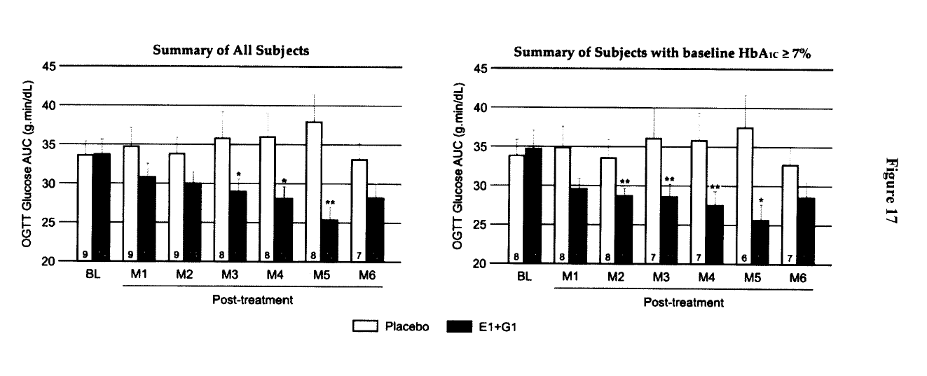

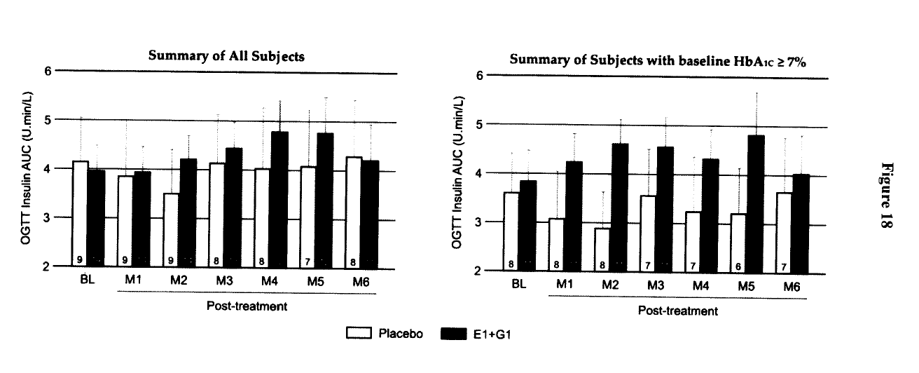

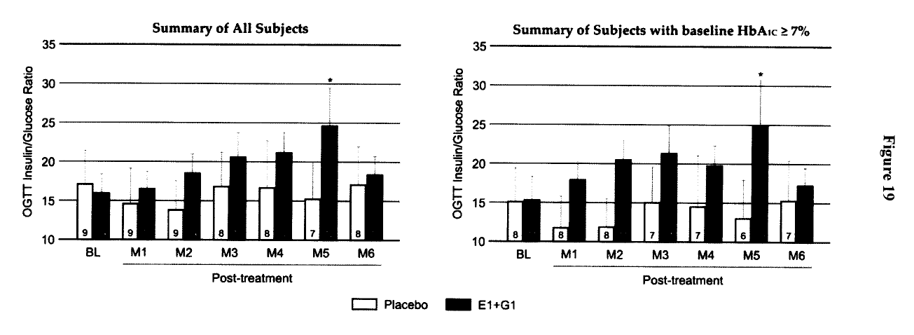

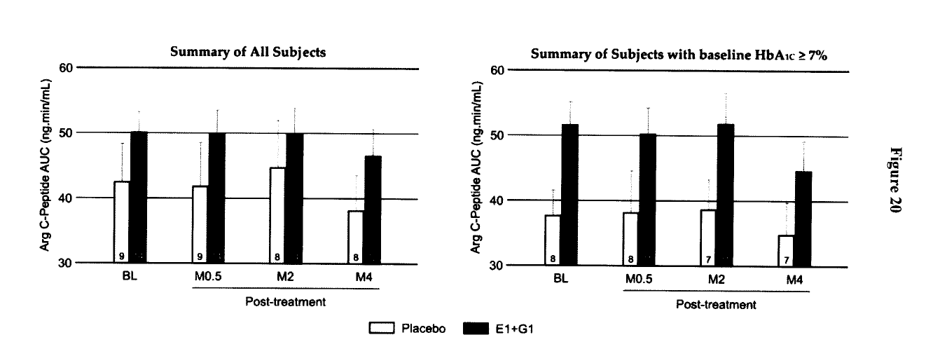

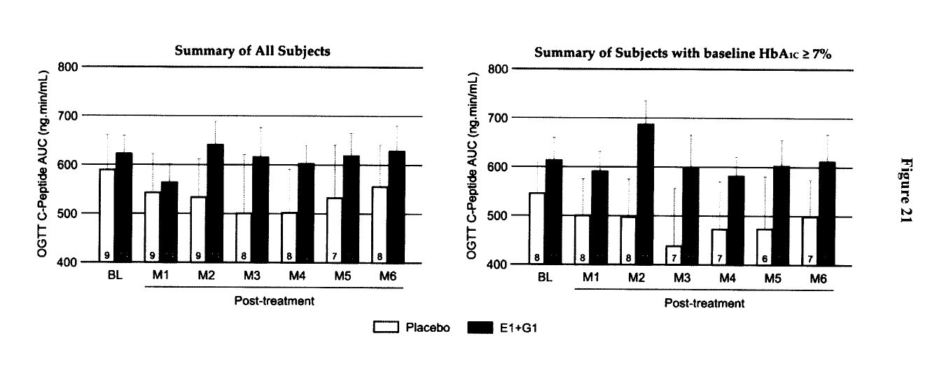

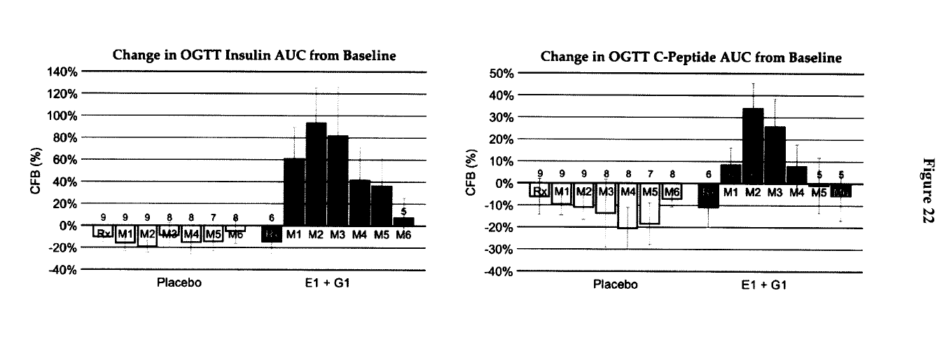

The analyses of Hemoglobin A_{1C} (HbA_{1c}) levels, fasting glucose levels, glucose levels, insulin levels, proinsulin levels, insulin usage and C-peptide levels are shown in FIGS. 13 to 22.

The treatment for 28 days showed positive data or trends in most diabetes efficacy parameters examined in Type 2 diabetes patients with baseline HbA1C levels greater than or equal to 7%. As shown in FIGS. 13 to 22, HbA1C levels significantly decreased from baseline by an average of 0.43%, 0.94% (p=0.0176), 1.09% (p=0.0156), 1.12% (p=0.0410), 1.21% (p=0.0469), and 1.14% in months 1, 2, 3, 4, 5 and 6 post-treatment, respectively, while HbA1C levels in placebo patients decreased by an average of 0.08% in month 2 post-treatment and increased by 0.40% (month 3), 0.64% (month 4) and 1.00% (month 5) post-treatment. Fasting glucose levels were reduced with an average decrease of 36 mg/dL (p=0.0195) at post-treatment month 5 compared to an increase of 17.2 mg/dL in the placebo patients. Glucose tolerance (OGTT glucose AUC) improved progressively in months 2, (p=0.0088), 3 (p=0.0049), 4 (p=0.0098) and 5 (p=0.0215) post-treatment. Insulin levels (OGTT insulin AUC) showed a positive trend in months 1, 2, 3, 4, and 5 post-treatment (data did not show significance). Insulin to glucose ratio showed a positive trend in months 1, 2, 3, and 4 and a significant increase at month 5 (p=0.0469) post-treatment.

OGTT C-peptide levels did not change significantly in the treated group although they did appear to be higher relative to placebo. In patients that had an HbA1C decrease greater than 1% during the first 3 months post-treatment, OGTT insulin and C-peptide levels increased by 82-93% and 26-34% in months 2 and 3 post-treatment, respectively.

The clinical data demonstrated that the treatment improved glucose control for Type 2 diabetes patients using metformin with/without thiazolidinediones (TZD). Patients with baseline HbA1C levels (a measure of blood glucose control over time) greater than or equal to 7% showed improvements in multiple important measures of blood glucose control including HbA1C, fasting blood glucose, glucose levels, and insulin levels. The reductions in the HbA1C levels shown in this trial were consistent with observed reductions in fasting blood glucose as well as improvements in glucose tolerance and increases in insulin levels.

### Summary

The results from the Phase IIa clinical trial indicated that 4-weeks of daily treatments with a gastrin-based therapy, E1-I.N.T.™ (E1 and G1), showed sustained reductions in blood glucose control parameters, haemoglobin A1C (HbA1c), for 6 months post-treatment. The Type 2 diabetes patients enrolled in this study were using metformin with/without thiazolidinediones (TZDs). Achieving sustained improvements in glucose control for many months post-treatment following a 4-week therapy is unprecedented in Type 2 diabetes. The improvements in HbA1c correlated with changes in multiple other clinical parameters suggesting that gastrin-based therapies, and specifically E1-I.N.T.™, have the potential to improve beta cell function and re-engage the body's natural mechanism to regulate glucose.

Analysis of efficacy parameters was performed on Type 2 diabetes patients with HbA1c levels of equal to or greater than 7% prior to treatment. In the E1-I.N.T.™ treated group of patients, the mean HbA1c level was reduced by 0.94% to 1.21% vs. baseline levels in months 2 to 6 post-treatment. More specifically, the mean HbA1c level among treated patients was reduced 0.43%, 0.94% (p<0.05), 1.09% (p<0.05), 1.12% (p<0.05), 1.21% (p<0.05), and 1.14% in months 1, 2, 3, 4, 5, and 6 post-treatment. In contrast, the mean HbA1c levels of the placebo group ranged from a reduction of 0.1% to an increase of 1.0% over the same period. In addition to the HbA1c reductions, the data demonstrated decreases in fasting blood glucose levels as well as improvements in glucose tolerance over a six month period following treatment. Trends in increased insulin levels as measured with an oral glucose tolerance test were also observed, particularly in patients where the HbA1c levels decreased over 1% with the therapy. These data are consistent with increased beta cell function observed in diabetes animal models where a short treatment with E1-I.N.T.™ resulted in a sustained increase in beta cell mass and function. These clinical improvements, including HbA1c reductions >1% in patients six month post-treatment, highlight that this therapy can provide patients significant clinical benefits in excess of 6 months.

There were no serious adverse events noted during the study. The most common adverse events reported by patients receiving E1-I.N.T.™, were nausea, diarrhea, headaches and vomiting which were generally mild to moderate in nature. The vast majority of these adverse events were reported during the treatment period with the occurrence of adverse events in the post-treatment phase being similar in both the treated and placebo groups.

### Example 3

### G1 Clinical Studies

### Study Drug—G1

G1 is a synthetic human peptide consisting of 17 amino acids (aa) and is the same length as the native gastrin. G1 contains a single aa change at position 15, where leucine replaces methionine to enhance molecular stability. G1 is much more stable than native gastrin to oxidative processes, and has full biological activity. The amino acid sequence of G1 is as follows:

| | |
|---|---|
| Single Letter Format: | |
| Pyr*-GPWLEEEEEAYGWLDF | [SEQ ID NO. 4] |
| Three Letter Format | |

1 Pyr* gly pro trp leu glu glu glu glu glu
11 ala tyr gly trp leu asp phe stop
Where *Pyr = Pyr-OH = pGlu = Glp =
pyroglutamic acid (cyclic Glu)

G1 is synthesized by solid phase peptide synthesis by Fmoc (9-fluorenylmethyloxycarbonate) chemistry on a polydimethylacrylamide gel resin. The Fmoc chemistry approach results in more consistent coupling of aa under gentler conditions than traditional tBOC (t-butyloxycarbonyl) chemistry and offers the advantage of not having to use hydrofluoric acid to cleave the finished peptide from the resin. Each amino acid beginning with the C-terminal Phe is added sequentially using standard Fmoc chemistry. Each step of the synthesis is checked for completion by testing for a free amine group with the 1, 2, 4, Trinitrobenzenesulfonic acid (TNBSA) test (for the step where proline is added to the growing chain, chloroanil is substituted for TNBSA). After synthesis of the chain is complete, the resin is washed with diethyl ether and then dried to a constant weight under vacuum. The peptide chain is cleaved from the resin using NH₃ in a pressure vessel over 4-5 days. After cleavage from the resin, the side chain protecting groups are removed by 5% ethanedithiol in trifluoroacetic acid (TFA). The crude peptide is extracted with TFA then dried to a constant weight under vacuum. Purification of the peptide is carried out using chromatography and the pure peptide is isolated by lyophilization. The lyophilisate is stored at least −70° C. The lyophilisate is dissolved in PBS (50 mM sodium phosphate, pH 7.4, 100 mM sodium chloride) and 0.01% polysorbate 80 (vegetable based) at a concentration >4 mg/mL. After sterile filtration, the concentration of G1 is adjusted to 4 mg/mL with sterile vehicle (phosphate buffered saline (PBS) 0.01% polysorbate 80) and dispensed aseptically at 1.0 mL/vial into 3 mL glass vials. The vials are stoppered and crimp capped then stored at least −70° C.

G1 is supplied as a sterile, frozen solution in PBS with 0.01% vegetable based polysorbate 80.

### Clinical Studies

Two studies have been completed assessing G1 alone. Other studies have been conducted wherein G1 was administered in combination with E1 (See Examples 1 and 2).

### 1. Phase 1 Study (E1 or G1)

A Phase 1 single ascending dose study was conducted as part of a G1/E1 islet neogenesis development program. In this study, individual components of the combination therapy, G1 and E1 were evaluated. This study was a blinded, vehicle-controlled, dose escalation study with single sc administration of either G1 or E1 or vehicle control in healthy male subjects. The objective of the Phase 1 trial was to evaluate the safety, tolerability and PK profile of single escalating sc doses of G1 or E1. A total of 9 subjects received a single dose of G1 at 3 different dose levels. Three (3) subjects received 3.0 μg/kg, 3 subjects received 10 μg/kg and 3 subjects received 30 μg/kg. For a 70 kg individual these μg/kg doses correspond to approximately 0.2 mg to 2 mg. A total of 7 subjects received sc doses of vehicle control.

### Conclusions

Single doses of G1 ranging from 3 μg/kg-30 μg/kg (approximately 0.2 mg to 2 mg in a 70 kg individual) were well tolerated and safe in a healthy male population.

### 2. Phase 1 Study (G1)

A Phase 1, single-center, randomized, placebo controlled, single-dose (SAD) study was conducted to evaluate the safety, tolerability and PK of G1 in healthy subjects. The study was conducted at a Phase 1 facility in Canada. Three different doses/dosing regimens of G1 were evaluated in three different subject cohorts so that each subject received only one dose level of study drug on a single study day. Subjects in Cohorts 1 and 2 received the same total daily dose (4 mg). Subjects in Cohort 1 received this dose as a single morning dose whereas subjects in Cohort 2 received this as a split dose; receiving 2 mg in the morning and 2 mg in the evening. Subjects in Cohort 3 received a single morning dose of 3 mg. Twenty-four healthy males were randomly assigned in a 3:1 fashion to receive either G1 or vehicle control (18 subjects received active drug and 6 subjects received vehicle control).

Safety was assessed through the reporting of AEs, clinical laboratory test results, physical examination findings, and vital signs measurements including supine and standing blood pressure and HR, and 12-lead ECG readings.

### Conclusions

The study drug G1 at doses of 2 mg bid, 3 mg and 4 mg were safe and well-tolerated.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein, or referenced in such documents are incorporated by reference in their entirety to the same extent as if each individual publication, patent or patent application was specifically and individually indicated to be incorporated by reference in its entirety. The citation of any reference herein is not an admission that such reference is available as prior art to the instant invention.

What is claimed is:

1. A pharmaceutical composition for treating diabetes in a patient with baseline HbA1c levels greater than about 5%, 6%, 7%, 8%, 9% or 10% comprising at least one gastrin compound in therapeutically effective amounts to provide beneficial effects for at least 1 to 6 months post treatment, and a pharmaceutically acceptable carrier, excipient, or vehicle, wherein the beneficial effects comprise control of haemoglobin A1c (HbA1c), fasting blood glucose, glucose levels and/or insulin levels.

2. A pharmaceutical composition according to claim 1 wherein the beneficial effects comprise a decrease in HbA1c levels by at least about 0.4%, 0.5%, 0.6%, 0.9%, 0.93%, 0.94%, 0.97%, 0.98%, 1%, 1.05%, 1.09%, 1.1%, 1.12%, 1.14%, 1.15%, 1.18%, 1.21%, 1.2%, 1.5%, or 2% post treatment in a Type 2 diabetes patient.

3. A pharmaceutical composition according to claim 1 wherein the gastrin compound is a gastrin or gastrin analogue.

4. A pharmaceutical composition according to claim 3 wherein the gastrin compound is gastrin-17(leu) of SEQ ID NO. 4

5. A method for preventing and/or treating diabetes in a subject with baseline HbA1c levels greater than about 5%, 6%, 7%, 8%, 9% or 10% comprising administering to the subject a therapeutically effective amount of at least one gastrin compound to control hemoglobin A1c (HbA1c), fasting blood glucose, glucose levels and/or insulin levels for at least about 1 to 6 months post treatment.

6. (canceled)

7. A method according to claim 5, wherein the therapeutically effective amount decreases HbA1c levels by at least about 0.1%, 0.25%, 0.4%, 0.43%, 0.45%, 0.5%, 0.6%, 0.75%, 0.8%, 0.85%, 0.9%, 0.93%, 0.94%, 0.95%, 0.97%, 0.98%, 1%, 1.05%, 1.09%, 1.1%, 1.12%, 1.14%, 1.15%, 1.18%, 1.21%, 1.2%, 1.5%, or 2%; increases insulin levels by at least about 50%, 70%, 75%, 80%, 90%, or 95%, and/or increases C-peptide levels by at least about 15%, 20%, 25%, 26%, 30%, 34%, 35%, 40%, 50%, 60%, 70%, 80%, or 90%

8. A method according to claim 5 wherein the therapeutically effective amount decreases HbA1c levels by at least about 0.1%, 0.25%, 0.4%, 0.43%, 0.45%, 0.5%, 0.6%, 0.75%, 0.8%, 0.85%, 0.9%, 0.93%, 0.94%, 0.95%, 0.97%, 0.98%, 1%, 1.05%, 1.09%, 1.1%, 1.12%, 1.14%, 1.15%, 1.18%, 1.21%, 1.2%, 1.5%, or 2%.

9. A method according to claim 1, wherein the subject suffers from Type 2 diabetes.

10. A method according to claim 9 wherein the subject is additionally receiving one or more glucose lowering agent and an insulin sensitivity enhancer.

11. A method for the potentiation of a glucose lowering agent and/or and an insulin sensitivity enhancer in the treatment of Type 2 diabetes in a subject comprising co-administering to the subject therapeutically effective amounts of at least one gastrin compound with the glucose lowering agent and/or insulin sensitivity enhancer.

12. A method according to claim 11, wherein the glucose lowering agent and/or insulin sensitivity enhancer are a metformin and/or a thiazolidinediones (TZD).

13. A method according to claim 11, wherein the glucose lowering agent is a metformin.

14. A method according to claim 1, wherein the gastrin compound is a gastrin or gastrin analogue.

15. A method according to claim 14 wherein the gastrin compound is gastrin-17(leu) of SEQ ID NO. 4

16. A method according to claim 1, wherein the gastrin compound is administered for about 2 to 6 weeks and treatment is stopped for about 6 to 9 months following administration of the gastrin compound.

17. (canceled)

18. (canceled)

19. (canceled)

20. (canceled)

21. A kit for carrying out a method according to claim 5.


SEQUENCE LISTING (16 sequences)

SEQ ID NO: 1 - protein, 34 aa, Homo sapiens
  MISC_FEATURE (1)..(1): Xaa = pyroglutamate, Gln or no amino acid residue
XLGPQGPPHLVADPSKKQGPWLEEEEEAYGWMDF

SEQ ID NO: 2 - protein, 34 aa, Artificial
  Synthetic Construct
  MISC_FEATURE (1)..(1): Xaa = pyroglutamate, Gln or no amino acid residue
XLGPQGPPHLVADPSKKQGPWLEEEEEAYGWLDF

SEQ ID NO: 3 - protein, 17 aa, Homo sapiens
  MISC_FEATURE (1)..(1): Xaa = pyroglutamate or no amino acid residue
XGPWLEEEEEAYGWMDF

SEQ ID NO: 4 - protein, 17 aa, Artificial
  Synthetic Construct
  MISC_FEATURE (1)..(1): Xaa = pyroglutamate or no amino acid residue
XGPWLEEEEEAYGWLDF

SEQ ID NO: 5 - protein, 101 aa, Homo sapiens
MQRLCVYVLIFALALAAFSEASWKPRSQQPDAPLGTGANRDLELPWLEQQGPASHHRRQLGPQGPPHLVADPSKKQGPWLEEEEEAYGWMDFGRRSAEDEN

SEQ ID NO: 6 - protein, 52 aa, Homo sapiens
DLELPWLEQQGPASHHRRQLGPQGPPHLVADPSKKQGPWLEEEEEAYGWMDF

SEQ ID NO: 7 - protein, 14 aa, Homo sapiens
WLEEEEEAYGWMDF

SEQ ID NO: 8 - protein, 6 aa, Homo sapiens
YGWMDF

SEQ ID NO: 9 - protein, 6 aa, Homo sapiens
YGWLDF

SEQ ID NO: 10 - protein, 6 aa, Artificial
  Fragment
YGWMDF

SEQ ID NO: 11 - protein, 6 aa, Artificial
  Fragment
YGWLDF

SEQ ID NO: 12 - protein, 10 aa, Homo sapiens
GAGAGAGAGA

SEQ ID NO: 13 - protein, 4 aa, Artificial
  Fragment
WMDF

SEQ ID NO: 14 - protein, 4 aa, Artificial
  Fragment
WLDF

SEQ ID NO: 15 - protein, 37 aa, Homo sapiens [sequence omitted]

SEQ ID NO: 16 - protein, 89 aa, Homo sapiens
  ref <308>: DATABASE ACCESSION NUMBER: NP_000406
  ref <309>: DATABASE ENTRY DATE: 2008-02-11
  ref <313>: RELEVANT RESIDUES IN SEQ ID NO: (1)..(89)
MGILKLQVFLIVLSVALNHLKATPIESHQVEKRKCNTATCATQRLANFLVHSSNNFGAILSSTNVGSNTYGKRNAVEVLKREPLNYLPL